(19) United States
(12) Patent Application Publication
(10) Pub. No.: US 2024/0101983 A1
(43) Pub. Date: Mar. 28, 2024

(54) PROGRAMMABLE RNA EDITING PLATFORM

(71) Applicant: NANYANG TECHNOLOGICAL UNIVERSITY, Singapore (SG)

(72) Inventors: Meng How TAN, Singapore (SG); Yuanming WANG, Singapore (SG); Kaiwen Ivy LIU, Singapore (SG); Kean Hean OOI, Singapore (SG)

(21) Appl. No.: 17/769,047

(22) PCT Filed: Oct. 19, 2020
(86) PCT No.: PCT/SG2020/050599

§ 371 Date: Apr. 14, 2022

(30) Foreign Application Priority Data
Oct. 18, 2019 (SG) 10201909733W

(51) Int. Cl. C12N 9/22; C12N 9/78

(52) U.S. Cl. CPC C12N 9/22; C12N 9/78; C07K 2319/00; C12N 2310/20

(57) ABSTRACT

The present invention relates to artificially designed polypeptides having RNA-targeting and editing activity, wherein said polypeptides are fusion proteins comprising a modified ADAR2 deaminase domain and a Cas family targeting moiety selected from deactivated Cas13b and CasRx. Further encompassed are methods for use and uses of these polypeptides, compositions comprising them and nucleic acids encoding them as well as methods for the manufacture of said polypeptides.

### CROSS-REFERENCE TO RELATED APPLICATIONS

This application claims the benefit of priority of Singapore Patent Application No. 10201909733W filed 18 Oct. 2019, the content of which being hereby incorporated by reference in its entirety for all purposes.

### FIELD OF THE INVENTION

The present invention lies in the technical field of RNA editing and specifically relates to artificially designed polypeptides having RNA-targeting and editing activity. Further encompassed are methods for use and uses of these polypeptides, compositions comprising them and nucleic acids encoding them as well as methods for the manufacture of said polypeptides.

### BACKGROUND OF THE INVENTION

Technologies that alter genetic information in the cell are valuable for multiple biomedical and biotechnological applications. Traditionally, the focus has been on introducing changes in the DNA and recent years have witnessed the rapid development of a wide array of genome engineering tools. In particular, CRISPR-associated nucleases such as Cas9 and Cas12a have been successfully used to manipulate the genome of many different living organisms. Furthermore, by fusing a natural or evolved deaminase domain to catalytically impaired Cas9, various groups have demonstrated that some types of point mutations can be efficiently corrected via base editing without the need for a double-stranded break.

Lately, there has been a growing interest in targeting RNA instead of DNA. When changes are introduced into RNA, they are transient and reversible as cells are continuously making new transcripts. Consequently, modifying the transcriptome offers at least two important advantages over editing the genome. First, RNA editing can be used to treat temporary conditions, such as pain or inflammation. It may also be used to stimulate tissue regeneration after injury. Second, RNA editing avoids the problems of permanent gene editing. Importantly, any potential off-target editing will not be fixed and propagated.

To achieve targeted RNA editing, researchers leverage on known RNA deaminases, specifically the ADAR and APOBEC family of enzymes. ADAR (adenosine deaminase acting on RNA type 2) enzymes convert adenosine (A) to inosine (1), which are recognized by cellular machineries as guanosines (G), whereas APOBEC (apolipoprotein B mRNA editing enzyme, catalytic polypeptide-like) enzymes convert cytidine (C) to uridine (U). Most efforts to date have focused on the use of ADARs to introduce A-to-G changes in selected RNA transcripts. Since ADARs are double-stranded RNA (dsRNA)-binding proteins, a stem structure containing the target site must be created, in order for the site to be edited. Furthermore, all the published strategies can be broadly separated into two categories. In the first category, endogenous ADARs already within the cell are recruited to the target site for it to be edited. The recruitment can be accomplished using long (greater than 100 nucleotides) or heavily chemically modified antisense oligonucleotides. In the second category, an engineered ADAR enzyme or its catalytic domain is ectopically expressed in the cell, with the modification designed to enable the deaminase to be recruited to a desired target site. This modification includes fusing ADAR to a λN peptide (Montiel-Gonzalez et al. (2013), Proc Natl Acad Sci USA 110, 18285-18290, doi:10.1073/pnas.1306243110 (2013), a SNAP tag (Vogel, P. et al. (2018) Nat Methods 15, 535-538, doi:10.1038/s41592-018-0017-z; Schneider et al. (2014) Nucleic acids research 42, e87, doi:10.1093/nar/gku272), a RNA-binding protein with a well-characterized substrate such as MS2 (Katrekar, D. et al. (2019) Nat Methods 16, 239-242, doi:10.1038/s41592-019-0323-0), or an inactive CRISPR-associated nuclease from the Cas13 family (Cox, D. B. T. et al. (2017) Science 358, 1019-1027, doi:10.1126/science.aaq0180).

Existing technologies for targeted RNA editing in mammalian cells have various problems. For example, one could have high on-target efficiency but has poor specificity, while another could have good specificity but bad on-target efficiency. More specifically, methods that rely on endogenous ADAR have various problems. First, their performance cannot be controlled as it is dependent on the expression level of the endogenous ADARs, which can be highly context-dependent. Second, endogenous ADAR also subjected to intracellular regulation in unexpected ways. For example, in muscle cells, endogenous ADAR protein level is very low due to degradation by high levels of AIMP2. Third, in the LEAPER method (Qu, L. et al. (2019) Nat Biotechnol 37, 1059-1069, doi:10.1038/s41587-019-0178-z), the guide RNAs used (called arRNAs) have to be longer than 100 base pairs, which has the potential to activate the innate immune response (e.g. via MDA5). Also, LEAPER suffers from a big trade-off between on-target efficiency and off-targeting editing. Fourth, in the RESTORE method (Merkle, T. et al. (2019) Nat Biotechnol 37, 133-138, doi:10.1038/s41587-019-0013-6), the guide RNA used to recruit endogenous ADARs has to contain extensive chemical modifications. Hence, this method is unlikely to be useful to most researchers. Fifth, there are many RNA species in the cell that are highly structured. These methods only rely on base pairing between an exogenously introduced guide RNA and a target (with no additional protein introduced). Consequently, it is unclear how well they will work for highly structured targets.

Due to the limitations above, the ideal situation is still to introduce an exogenous ADAR deaminase that can be readily programmed to target a specific site. The best system reported to date is the REPAIR (RNA Editing for Programmable A to I Replacement) platform, which relies on an inactive Cas13b (deactivated Cas13b or dCas13b) fused to human ADAR2 at the C-terminus (Cox et al., supra). Cas13 is a programmable single-effector RNA-guided ribonuclease belonging to the Type IV CRIPSR-Cas system. However, REPAIR suffers from a trade-off between efficiency and specificity. There is thus still need in the art to expand upon the original REPAIR concept and develop a technology that is both highly efficient and highly specific.

### SUMMARY OF THE INVENTION

The inventors of the present invention found that the activity of the deaminase domain (dd) of RNA Adenosine Deaminase 2 (ADAR2) can be tuned towards less off-target activity while retaining high on-target activity by introducing mutations that replace positively charged amino acid residues in the ADAR2 protein that may increase “stickiness” towards generic RNA by other residues. Off-target activity could be further lowered by replacing the Cas13b scaffold by CasRx, including CasRx variants such as CasRx K942L. Additionally, the design of the guide RNA for the dCasRx (deactivated CRISPR-associated Rx) ADAR2dd fusion could be further optimized with respect to length and sequence. Fusion of ADAR2 to certain internal sites in CasRx/Cas13b resulted in similar on-target efficiencies but even lower off-target editing than the original C-terminus fusion construct. Off-target activity could further be lowered by splitting the CasRx domain and rearranging the fragments. Certain cis off-targets could be eliminated by modifying the gRNA such that guanosines were put opposite adenosines that were wrongly edited.

Based on the above findings, in a first aspect, the present invention thus relates to an isolated polypeptide comprising or consisting of
(1) a first polypeptide domain comprising an amino acid sequence that
   (i) shares at least 70, preferably at least 80, more preferably at least 90%, most preferably at least 95% sequence identity with the amino acid sequence set forth in SEQ ID NO:1 over its entire length; and
   (ii) comprises the amino acid substitution 173Q using the positional numbering of SEQ ID NO:1; and, optionally,
   (iii) comprises amino acid substitutions at any one or more of the positions corresponding to positions 145, 33, 34, 36, 139, 140, 142, 143, 154, 155, 156, 158, 159, 160, 162, and 164 of SEQ ID NO:1 (hADAR2dd);
(2) a second polypeptide domain comprising an amino acid sequence that shares at least 70, preferably at least 80, more preferably at least 90%, most preferably at least 95% sequence identity with
   (i) the amino acid sequence set forth in SEQ ID NO:2 over its entire length (dCasRx) and comprises the amino acid substitutions 239A, 244A, 858A, and 863A using the positional numbering of SEQ ID NO:2; or
   (ii) the amino acid sequence set forth in SEQ ID NO:3 over its entire length (dCas13b) and comprises the amino acid substitutions 133A and 1058A, using the positional numbering of SEQ ID NO:3;
      wherein the first polypeptide domain is fused to the second polypeptide domain or inserted into the second polypeptide domain;
      with the proviso that if the second polypeptide domain comprises an amino acid sequence that shares at least 70, preferably at least 80, more preferably at least 90%, most preferably at least 95% sequence identity with the amino acid sequence set forth in SEQ ID NO:3 and comprises the amino acid substitutions 133A and 1058A, using the positional numbering of SEQ ID NO:3, the first polypeptide domain does not have the amino acid sequence of SEQ ID NO:1 with the amino acid substitution 1730 in combination with one of 33E, 36L, 140G/S/E, 158D, 159E, 1600, and 162E.

In a second aspect, the present invention relates to an isolated polypeptide comprising or consisting of
(1) a fragment of a first polypeptide domain, wherein said first polypeptide domain has an amino acid sequence that
   (i) shares at least 70, preferably at least 80, more preferably at least 90%, most preferably at least 95% sequence identity with the amino acid sequence set forth in SEQ ID NO:1 over its entire length; and
   (ii) comprises the amino acid substitution 173Q using the positional numbering of SEQ ID NO:1; and, optionally,
   (iii) comprises amino acid substitutions at any one or more of the positions corresponding to positions 33, 34, 36, 139, 140, 142, 143, 145, 154, 155, 156, 158, 159, 160, 162, and 164 of SEQ ID NO:1;
   wherein
   (a) said fragment is a C-terminal fragment of 230-239 amino acids in length and comprises at least 230 amino acids corresponding to positions 156 to 385 of SEQ ID NO:1; or
   (b) said fragment is an N-terminal fragment of 146-155 amino acids in length and comprises at least 146 amino acids corresponding to positions 1 to 146 of SEQ ID NO:1; and
(2) a second polypeptide domain, wherein the second polypeptide domain comprises an amino acid sequence that shares at least 70, preferably at least 80, more preferably at least 90%, most preferably at least 95% sequence identity with the amino acid sequence set forth in SEQ ID NO:2 over its entire length and comprises the amino acid substitutions 239A, 244A, 858A, and 863A and optionally 940L using the positional numbering of SEQ ID NO:2; and
wherein, if the first polypeptide domain fragment is an N-terminal fragment, it is fused to the C-terminus of the second polypeptide domain, or if the first polypeptide domain fragment is a C-terminal fragment, it is fused to the N-terminus of the second polypeptide domain.

In a third aspect, the invention relates to an isolated polypeptide comprising or consisting of
(1) a fragment of a first polypeptide domain, wherein said first polypeptide domain has an amino acid sequence that
   (i) shares at least 70, preferably at least 80, more preferably at least 90%, most preferably at least 95% sequence identity with the amino acid sequence set forth in SEQ ID NO:1 over its entire length; and
   (ii) comprises the amino acid substitution 1730 using the positional numbering of SEQ ID NO:1; and, optionally,
   (iii) comprises amino acid substitutions at any one or more of the positions corresponding to positions 33, 34, 36, 139, 140, 142, 143, 145, 154, 155, 156, 158, 159, 160, 162, and 164 of SEQ ID NO:1;
   wherein
   (a) said fragment is a C-terminal fragment of 230-239 amino acids in length and comprises at least 230 amino acids corresponding to positions 156 to 385 of SEQ ID NO:1; or
   (b) said fragment is an N-terminal fragment of 146-155 amino acids in length and comprises at least 146 amino acids corresponding to positions 1 to 146 of SEQ ID NO:1; and
(2) a second polypeptide domain, wherein the second polypeptide domain comprises an amino acid sequence that shares at least 70, preferably at least 80, more preferably at least 90%, most preferably at least 95% sequence identity with the amino acid sequence set forth in SEQ ID NO:3 over its entire length and comprises the amino acid substitutions 133A and 1058A using the positional numbering of SEQ ID NO:3; and
wherein, if the first polypeptide domain fragment is an N-terminal fragment, it is fused to the C-terminus of the second polypeptide domain, or if the first polypeptide domain fragment is a C-terminal fragment, it is fused to the N-terminus of the second polypeptide domain.

In another aspect, the invention is directed to a composition comprising at least two polypeptides, wherein the first polypeptide is the isolated polypeptide of the second aspect of the invention and the second polypeptide is the isolated polypeptide of the third aspect of the invention, wherein if the first polypeptide comprises the N-terminal fragment of the first polypeptide domain, the second polypeptide comprises the C-terminal fragment of the first polypeptide domain, or wherein if the first polypeptide comprises the C-terminal fragment of the first polypeptide domain, the second polypeptide comprises the N-terminal fragment of the first polypeptide domain.

A still further aspect of the invention is directed to the composition comprising the isolated polypeptide of the first aspect of the invention or the above composition of the invention and further comprising a guide RNA (gRNA) molecule.

In still another aspect, the invention relates to a pharmaceutical composition comprising the isolated polypeptide of the invention or the composition of the invention and one or more of diluents, stabilizers, excipients and carriers.

Another aspect relates to the isolated polypeptide of the invention or the composition of the invention for use as a pharmaceutical.

Also encompassed is the use of the isolated polypeptide of the invention or the composition of the invention for targeted RNA editing, including in vitro or in vivo RNA editing.

In a still further aspect, the invention is directed to a method for targeted editing of the RNA of a cell, comprising introducing into said cell the isolated polypeptide of the invention or the composition of the invention.

Another aspect relates to a method for the treatment or prevention of SARS-CoV-2 infection, pain (pain management), or epidermolysis bullosa comprising administering a therapeutically or prophylactically effective amount of a composition of the invention to a subject in need thereof.

A still further aspect also relates to nucleic acid molecules encoding the polypeptides described herein, as well as a vector containing such a nucleic acid, in particular a copying vector or an expression vector.

In a further aspect, the invention is also directed to a host cell, preferably a non-human host cell, containing a nucleic acid as contemplated herein or a vector as contemplated herein.

A still further aspect of the invention is a method for manufacturing a polypeptide as described herein, comprising culturing a host cell contemplated herein; and isolating the polypeptide from the culture medium or from the host cell.

### BRIEF DESCRIPTION OF THE DRAWINGS

FIG. 1. Residues in ADAR2 that are close to and may interact with the bound RNA duplex A are shown in the complex structure of ADAR2 and bound RNA duplex.

FIG. 2. Residues shown in FIG. 1 were mutated and the effect of the mutations tested, either individually or in various combinations, on the performance of the REPAIR platform using a luciferase recovery assay. (a) The luciferase reporter contains a nonsense mutation at W60 (SEQ ID NO:164). (b) The luciferase reporter contains a nonsense mutation at W219 (SEQ ID NO:167).

FIG. 3. An alternative Cas13 family member may be used as a scaffold for the ADAR2 deaminase domain. (a) ADAR2 (v1: E488Q only, v2: E488Q and T375G) was fused to the C-terminus of inactive CasRx (dCasRx) and the new CasRx-based enzymes were compared against the original Cas13b-based constructs in luciferase recovery assays. (b-d) The activity of dCasRx-v1 was evaluated when various spacer lengths and mismatch distances were used for the guide RNAs. Overall, it was found that 26 nt spacers worked just as well as, if not better than, 50 nt spacers. It was also found that the optimal mismatch distance for 26 nt spacers was between 7-15 nt.

FIG. 4. Tuning the design parameters of a dCasRx-based programmable RNA editing platform. (a) Keeping the mismatch distance fixed at 25 nt, while varying the spacer length subcellular localization of the enzyme, and the position of ADAR2 fusion. It was found that the platform only works in the nucleus. Furthermore, C-terminal fusion of the deaminase domain yields higher editing efficiencies than N-terminal fusion. (b-c) Three different linkers between dCasRx and ADAR2 were tested. Overall, when all the luciferase reporters are considered together, the XTEN linker gives significantly higher editing efficiencies than both the short (sequence: Gly-Ser; GS) and long GS (SEQ ID NO:57) linkers. (d) Two copies of ADAR2 at both termini of dCasRx did not give higher editing efficiencies than a single ADAR2 domain fused at the C terminus. (e) Fine tuning the spacer length. Anything shorter than 24 nt is detrimental to the performance of the platform. (f) Varying the identity of the nucleotide opposite the adenosine to be edited. A cytosine is highly favorable for editing. (g) Confirming the optimal guide RNA design with additional luciferase reporters.

FIG. 5. Validating the efficiency of dCasRx-based RNA editing with optimized design on genome-encoded transcripts, KRAS, PPIB and RAB7A. A range of mismatch distance based on the 7-15 nt window was tested. On all 4 sites, dCasRx-v1 is able to perform just as well as, if not better, than REPAIRv1.

FIG. 6. Different promising mutant ADAR2 deaminase domains were fused individually to the C-terminus of dCasRx and the efficiency and specificity of these constructs tested using the (a) W60X and (b) W219X luciferase reporters. All the mutations were able to reduce off-target editing, but at some expense of on-target activity.

FIG. 7. When the deaminase domain is fused at the C-terminus, it may have high flexibility to edit random RNAs independent of dCasRx binding. Without wishing to be bound by any particular theory, it was hypothesized that by fusing ADAR2 internally within dCasRx the freedom of the ADAR2 would be restricted only to the gRNA-target duplex. Hence, using available protein structures of CasRx orthologs, several alternative sites located on the external surface of CasRx that may be amenable to domain fusion as well were identified. 7 sites with varying distance to the gRNA-target duplex were chosen (In1: F406, In2: E689, In3: G655, In4: D338, In5: A376, In6: A878, In7: T558). The first four fusion positions (In1, In2, In3, and In4) will place the ADAR2 deaminase domain closer to the RNA duplex than the C-terminus.

FIG. 8. It was found that fusion of ADAR2 at three of these internal sites (In2, In3, In4) resulted in appreciable on-target activity and reduced off-target editing in the (a) W60X and (b) W219X luciferase recovery assays. Addition of mutations to the ADAR2 deaminase domain further enhanced the specificity of the enzyme in both the (c) W60X and (d) W219X assays.

FIG. 9. The findings in FIG. 8 were further validated with additional sites in (a) W104X and (b) W153X. The findings were consistent where dCasRx-v1 In2 fusion provides best on-target efficiency with lower off-target activity which can be improved further when specificity enhancing mutations were added to the ADAR2 domain.

FIG. 10. It was observed that at the guide RNA-target RNA interface, additional adenosines within the RNA duplex may be edited besides the intended site, termed cis off-targets. To eliminate the cis off-target editing, a guanosine was placed opposite each of these additional adenosines. Using the REPAIRv1 platform, A-G mismatches were introduced at unintended adenosines to guide RNAs targeting (a) KRAS and (b) PPIB. At less than two A-G mismatches, it was found that cis-off target editing is significantly reduced without affecting on-target editing. However, on-target efficiency is slightly reduced when two or more mismatches were introduced. The sequences shown in FIGS. 10a and b are set forth in SEQ ID NOS: 133-144, the target DNA sequences in SEQ ID NOS:145-149.

FIG. 11. The same strategy as shown in FIG. 10 was employed to the dCasRx-v1 platform to eliminate cis-off target activity. Shown here is an example from GAPDH. It was found that the decrease in on-target activity can be compensated when the guide RNA is extended. The sequences shown in FIG. 11 are set forth in SEQ ID Nos. 172-176.

FIG. 12. 81 nt Extended gRNA with mismatch distance of 11

FIG. 13. Extended gRNA with 81 nt with mismatch distance of 11, linked with another adjacent gRNA upstream

FIG. 14. 81 nt Extended gRNA with mismatch distance of 11, linked with another adjacent gRNA downstream

FIG. 15. 80 nt Extended gRNA with mismatch distance of 40, linked with another gRNA

FIG. 16. Luciferase reporter assay using extended gRNA. RNA editing rates using luciferase reporter assay show a higher editing rate when used with dCasRx-K942L. Editing rates were further increased when paired with extended gRNA (extended upstream and downstream) together with dCasRx-K942L.

FIG. 17. Chromatograms showing RNA editing levels. Higher RNA editing rates (as depicted by red box) using dCasRx paired with normal gRNA or dCasRx-K942L paired with extended gRNA (5′ extend for RAB11A, 3′ extend for RADX, middle mismatch for ACE2).

FIG. 18. A G mismatch ‘bulge’ was created in the gRNA to counter cis off targets that could be edited.

FIG. 19. Luciferase reporter assay for editing effect of different mismatch. RNA editing rates using luciferase reporter assay shows effect of mismatch on the opposite of targeted adenosine.

FIG. 20. Chromatograms showing cis off-target editing. Compared to normal gRNA, extended gRNA could cause some cis off-target editing, which could be decreased by introducing A-G mismatch in the potential off-target sites. On-target and off-target sites were depicted by solid and dotted boxes respectively.

FIG. 21. Extended gRNA by fusing dCas13b and dCasRx system and their respective gRNAs

FIG. 22. Extended gRNA by fusing dCas13b and dCasRx system and their respective gRNAs. The gRNAs are driven by their own respective promoters.

FIG. 23. Luciferase reporter assay using different constructs. Comparison of RNA editing levels using different constructs and gRNAs. Editing level is significantly higher using the extended gRNA system by fusion of dCas13b and dCasRx gRNAs in the cytoplasm.

FIG. 24. Chromatograms showing RNA editing levels using dCasRx, dCasRx and dCas13b fusion system. RNA editing levels using the extended gRNA fusing dCasRx and dCas13b system with the same promoter shows a higher editing rate in both the nucleus (NLS) and cytoplasm (NES), with editing in the cytoplasm further increase editing efficiency. The sequence shown is set forth in SEQ ID NO:177.

FIG. 25. The ADAR deaminase domain is split and each half fused to dCas13b and dCasRx. By using the extended gRNA system and their respective gRNAs, the ADAR deaminase domain will be active when they dimerize in close proximity.

FIG. 26. Site where the ADAR deaminase domain is split—at L464

FIG. 27. Luciferase reporter assay comparing split ADAR system and dCasRx. RNA editing improved significantly when using split ADAR with 50 gRNA length with 25 mismatch distance having the highest editing rates compared to longer gRNA lengths

FIG. 28. Luciferase reporter assay for off target. Off target effects (when transfected with a non-targeting gRNA) when using the split ADAR system is 11-fold lower than dCasRx

FIG. 29. Chromatograms showing RNA editing levels using split ADAR system. RNA editing levels (depicted by the red box) were improved when split ADAR system was used and off target effects reduced.

FIG. 30. Schematic showing 4 different constructs of the invention for further improvement of the editing activity of an internally fused ADAR system. The different protein fragments were rearranged or the linkers were extended in length. The rationale for constructs (2) and (3) was that it was found from the crystal structure of ADAR2dd that its N- and C-terminus are relatively far apart. When trying to fuse ADAR2dd internally of dCasRx, the N- and C-terminus are forced to come closer together, which may strain the deaminase domain. Hence, it was investigated how it performs if free from this strain. dCasRx-In4 (SEQ ID NO:154), dCasRx-In4-CN (SEQ ID NO:158), dCasRX-In4-CN2 (SEQ ID NO:162), dCasRx-In4-ex-XTEN (SEQ ID NO:163).

FIG. 31. Luciferase reporter assay evaluating the different strategies to improve the on-target activity and the specificity of an internally fused ADAR system: Off-target plot. Using a random non-targeting gRNA, it was observed that both the dCasRx-In4-CN (SEQ ID NO:158) and dCasRx-In4-CN-K942L (SEQ ID NO:160) give higher off-target editing than dCasRx-In4 (SEQ ID NO:154), but lower off-target editing than dCasRx-v1, i.e. a simple fusion of ADAR2dd at the C-terminus of dCasRx.

FIG. 32. Luciferase reporter assay evaluating the different strategies to improve the on-target activity and the specificity of an internally fused ADAR system: On-target plot. From the on-target plot (i.e. using a gRNA that correctly targets the intended site), it was observed that dCasRx-In4-CN (SEQ ID NO:158) gives higher activity than dCasRx-In4 (SEQ ID NO:154). Furthermore, the dCasRx K942L mutation (K940L using the positional numbering of SEQ ID NO:2), the construct (SEQ ID NO:160) performs even better. If the H145D mutation is additionally added to the ADAR2dd domain of dCasRx-In4-CN-K942L, the off-target activity becomes remarkably low, but at the same time, the on-target activity is still better than dCasRx-In4 and dCasRx-v1. dCasRx-In4-CN-K942L-H460D (SEQ ID NO:161), which has very low off-target, but also exhibits improved on-target activity is thus one of the preferred constructs of the invention.

FIG. 33. Chromatograms showing RNA editing levels at an off-target site in F11R, which contains a long double-stranded RNA structure. No off-target is observed in the best-performing construct (dCasRx-In4-CN-K942L-H460D; SEQ ID NO:161).

FIG. 34. Chromatograms showing RNA editing levels in ACE2 K353 and K31.RNA editing levels (depicted by the red box) using dCasRx(942L) and extended gRNA. The sequences shown are set forth in SEQ ID NO:178 and 179.

FIG. 35. Chromatograms showing RNA editing levels in TMPRSS2 S441.RNA editing levels (depicted by the red box) using dCasRx(942L) and normal gRNA. The sequence shown is set forth in SEQ ID NO:180.

FIG. 36. Chromatograms showing RNA editing levels in SCN4A K1244.RNA editing levels (depicted by the red box) using dCasRx(942L) and normal gRNA. The sequence shown is set forth in SEQ ID NO:181.

FIG. 37. Chromatograms showing RNA editing levels in KRT14 R125.RNA editing levels (depicted by the red box) using dCasRx(942L) and extended gRNA. The sequence shown is set forth in SEQ ID NO:182.

### DETAILED DESCRIPTION

The present invention is based on the inventors' identification of a novel RNA editing platform that uses the mutated deaminase domain (dd) of RNA Adenosine Deaminase 2 (ADAR2) in combination with a targeting moiety derived from a deactivated endonuclease of the CRISPR-associated (Cas) family of proteins, namely Cas13b or CasRx. The Cas domain uses a guide RNA to target a specific site in an RNA molecule. Once bound to the target, the ADAR2dd converts a target adenosine (A) to inosine (1), which is recognized by cellular machineries as guanosine. This means that an A-to-G change is introduced into the RNA. This approach overcomes drawbacks in existing technologies that suffer from non-specific activity of ADAR that results in binding of generic RNA and thus causes off-target editing. It was furthermore found that the new methods can be further optimized by altering the type and sequence of the targeting moiety, altering the guide RNA sequence, splitting the ADAR deaminase domain into two partial domains each bound to a separate targeting moiety that bind adjacently to each other to the target RNA, and combinations of all these modifications.

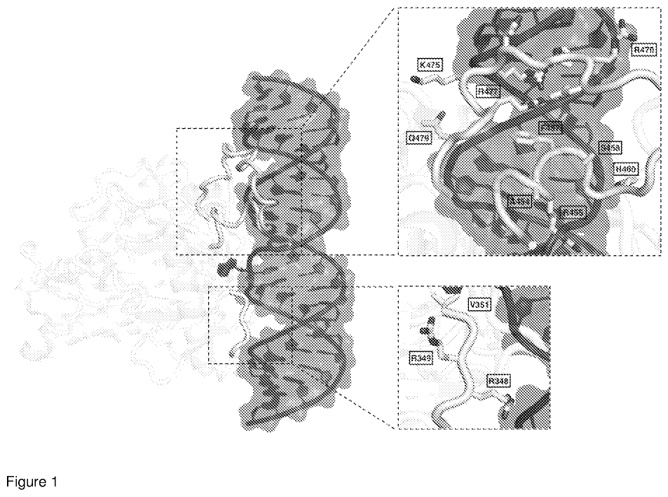

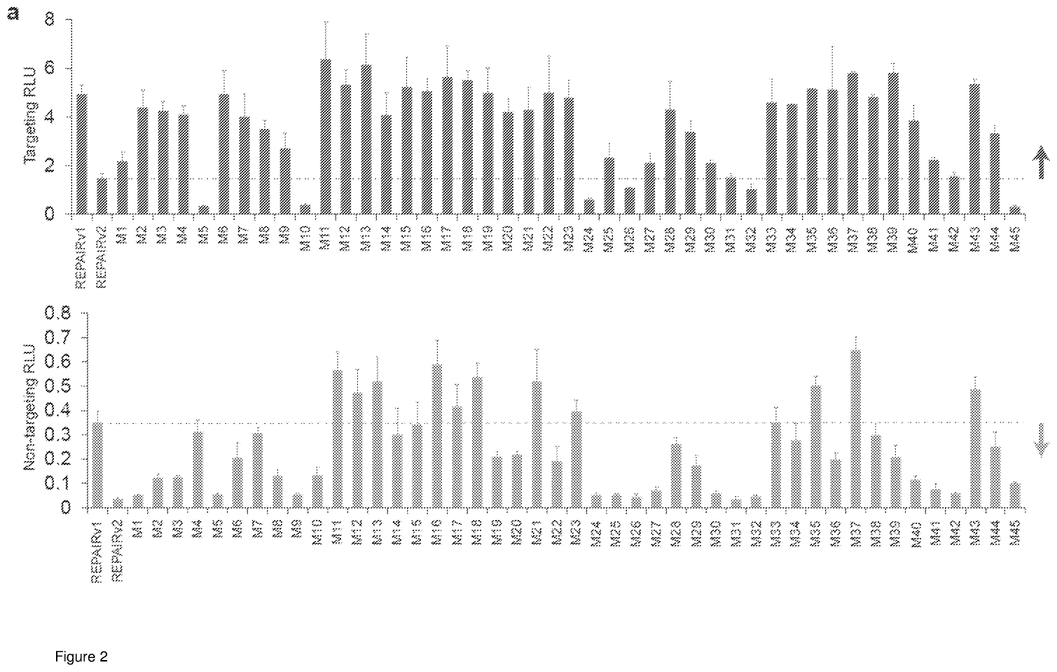

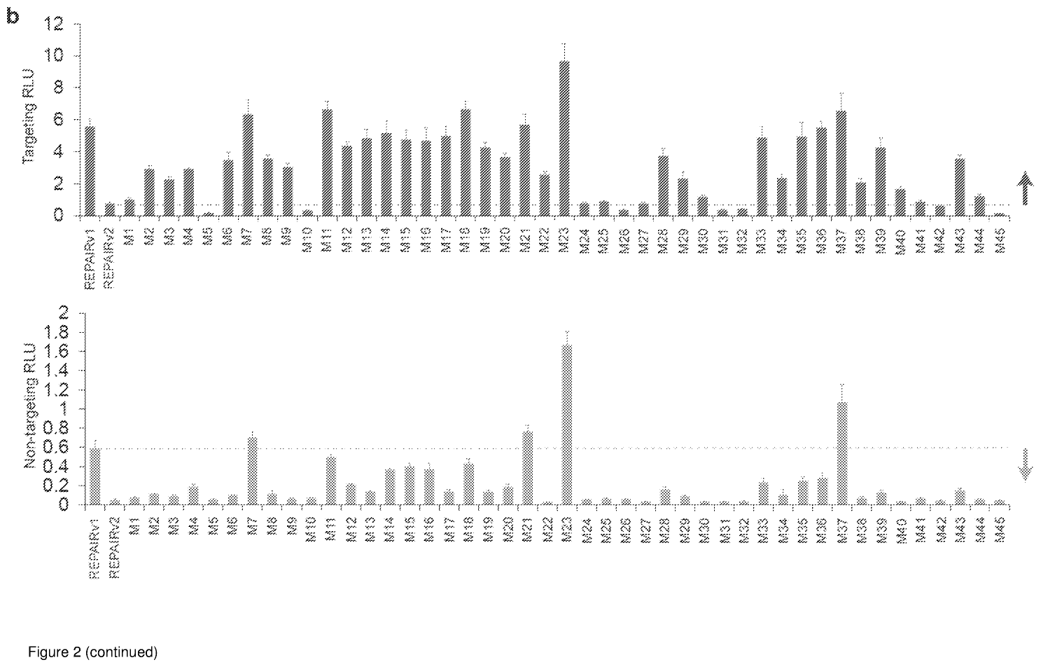

Specifically, the inventors of the present invention first found that the deaminase domain of ADAR2 might be excessively “sticky” and thus possess some non-specific ability to bind to generic RNA. Hence, since RNA is negatively charged due to its phosphate backbone, some positively charged amino acids in the ADAR2 protein were to be mutated. Hence, by examining the published crystal structure of the human ADAR2 deaminase domain, several positively charged residues that are close to the target RNA duplex were identified (see FIG. 1). Then 46 distinct human ADAR2 deaminase mutants (see Table 1) were tested in luciferase recovery assays and several promising mutations, such as H460D, that reduced off-target editing of dCas13b-ADAR2 but still maintained a reasonably good on-target efficiency were identified (see FIG. 2). In addition, since the cell is replete with double-stranded RNAs (dsRNAs), which form ideal substrates for ADAR enzymes, three such dsRNAs located in APOOL, F11R, and XIAP were deeply sequenced after human cells (HEK293T) were transfected with various dCas13b-ADAR constructs in the absence of any targeting guide RNA. Strikingly, all the enzymes tested, including the published REPAIRv2 platform, could edit some of the adenosines located within these dsRNAs (data not shown), thereby indicating that further optimization was required to improve the technology.

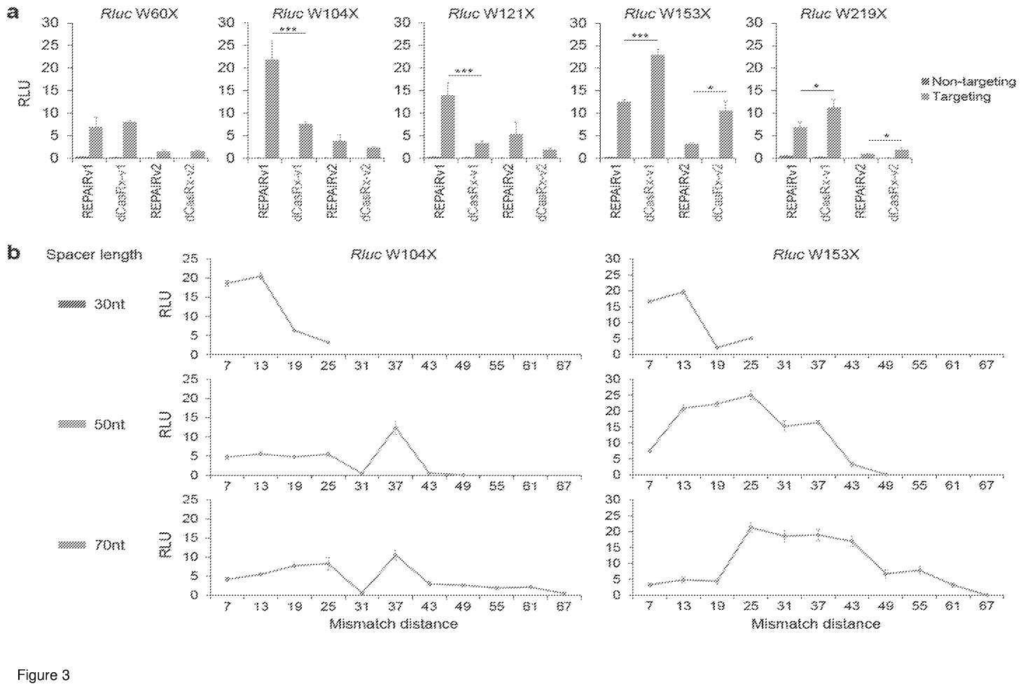

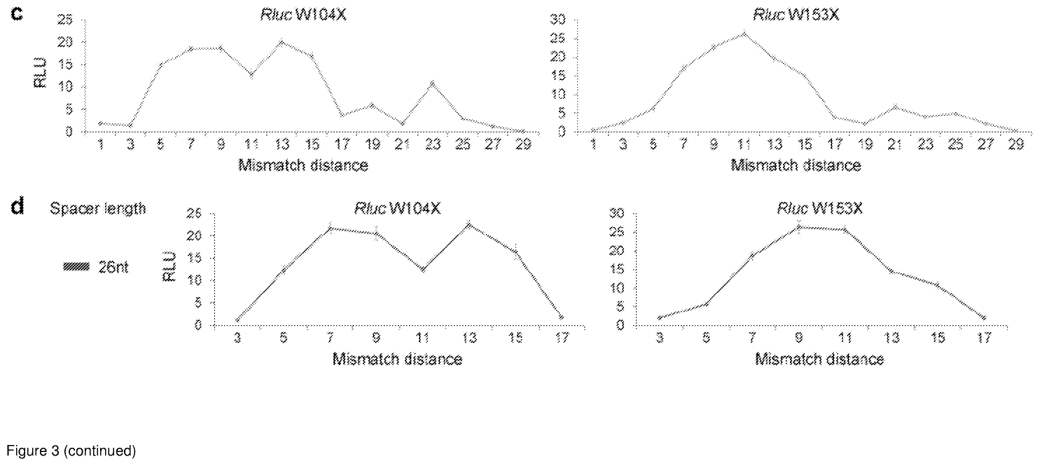

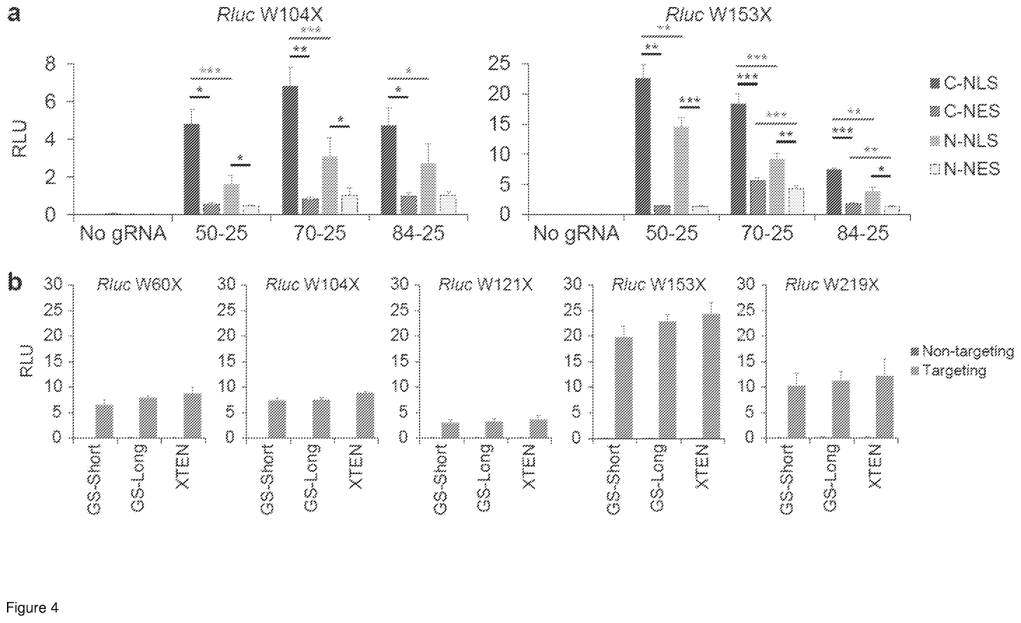

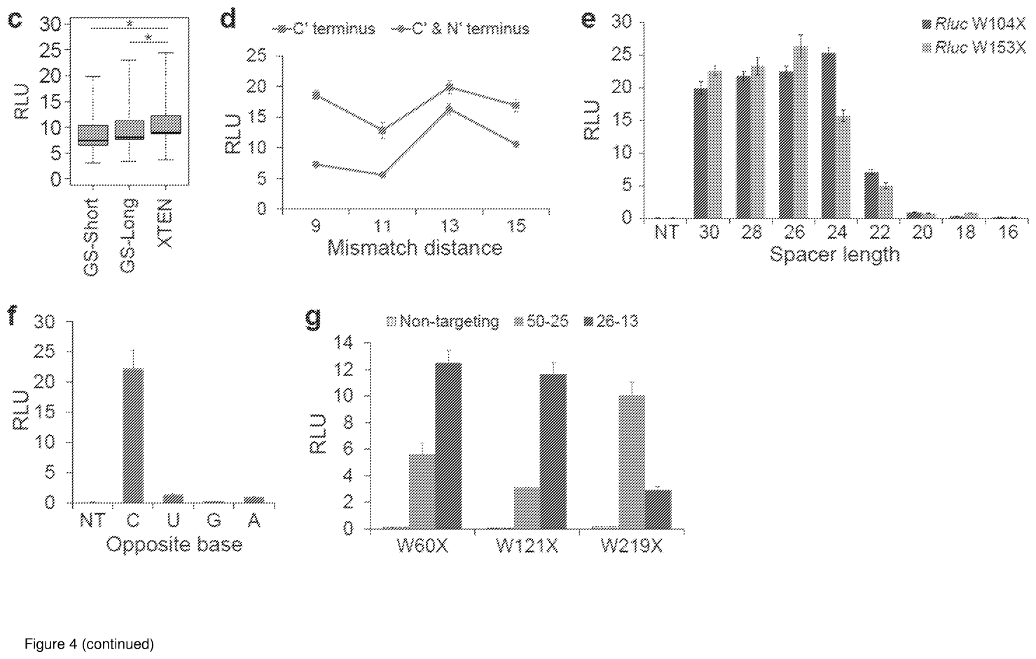

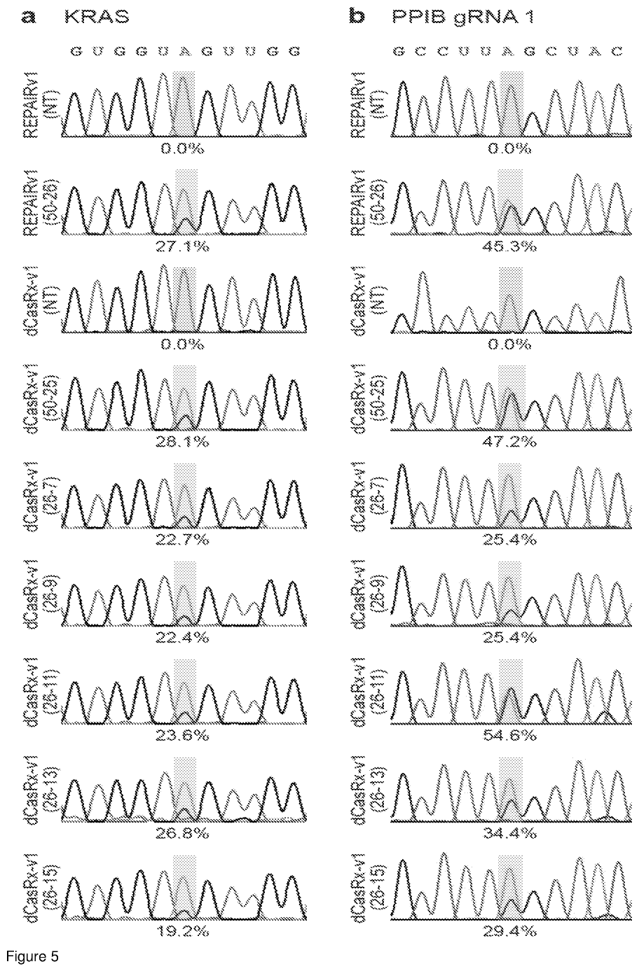

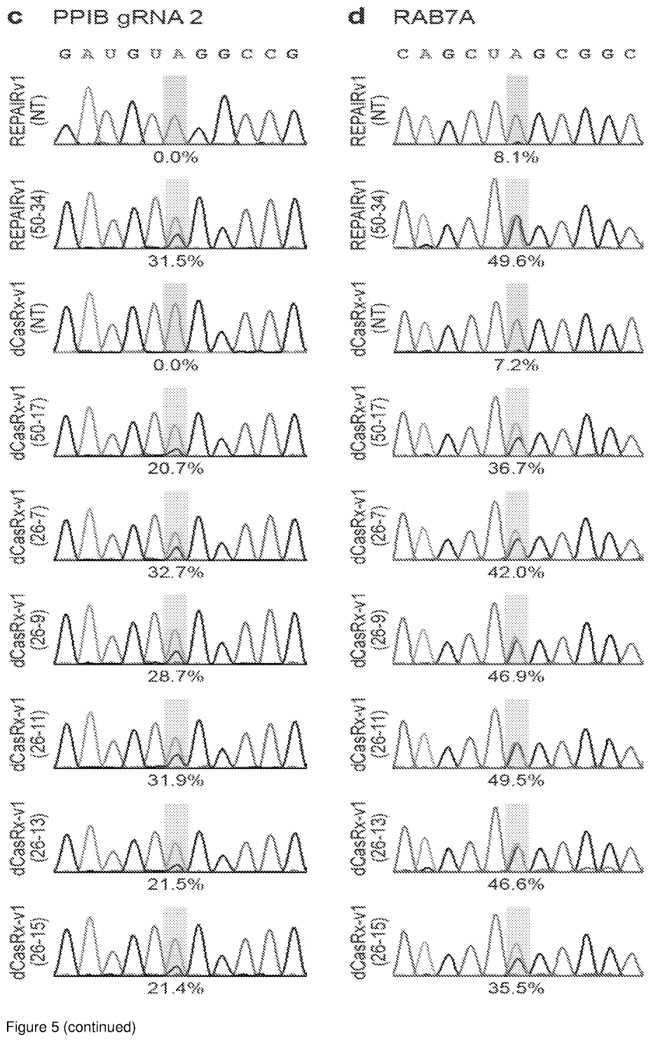

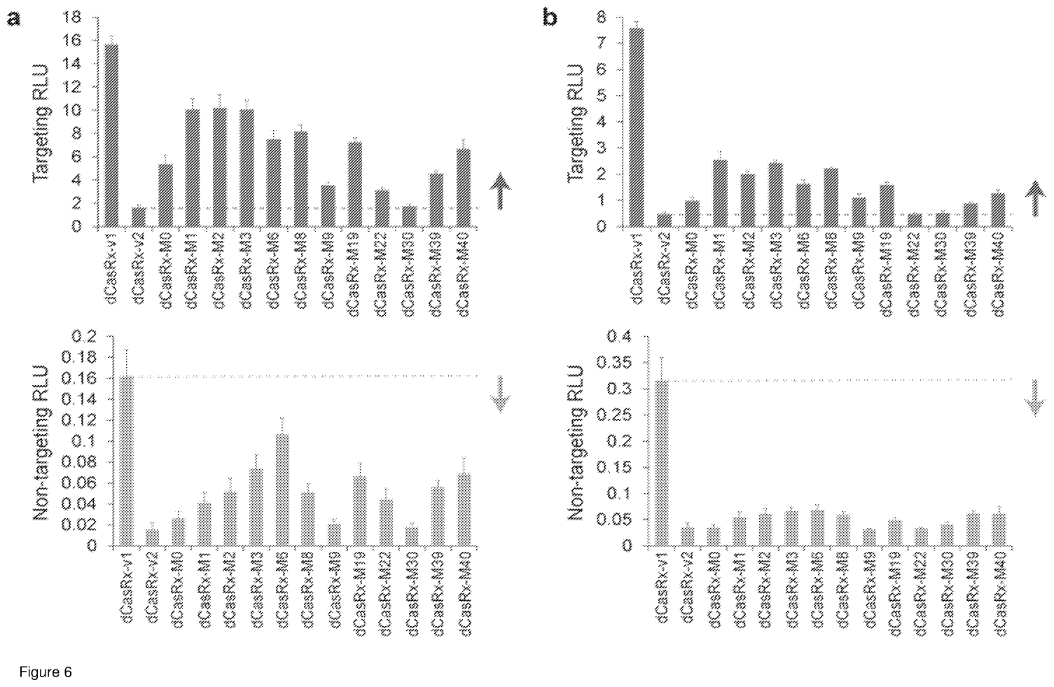

Secondly, another Cas13 family member, called CasRx, was used as a replacement scaffold instead of Cas13b. It was found that fusion of inactive CasRx (dCasRx) to ADAR2 can perform just as well as the known REPAIR platform that uses Cas13b with even lower off-targets in many cases (see FIG. 3). The design of the guide RNA for the dCasRx-ADAR2 fusion construct as well as the design of the editing enzyme itself was further optimized (see FIGS. 3 and 4). Interestingly, guide RNAs with spacers as short as 26 nt could recruit dCasRx-ADAR2 to target sites as efficiently as longer spacers in luciferase recovery assays. The design parameters were confirmed by targeting various endogenous genes (see FIG. 5). Moreover, it was demonstrated that the promising mutant ADAR2 domains that we identified earlier with dCas13b could also work well with dCasRx when they were fused at the C-terminus (see FIG. 6). Since many of the thus generated dCasRx-ADAR2 constructs still exhibited some off-target activity, although largely at low levels, further optimization steps were pursued.

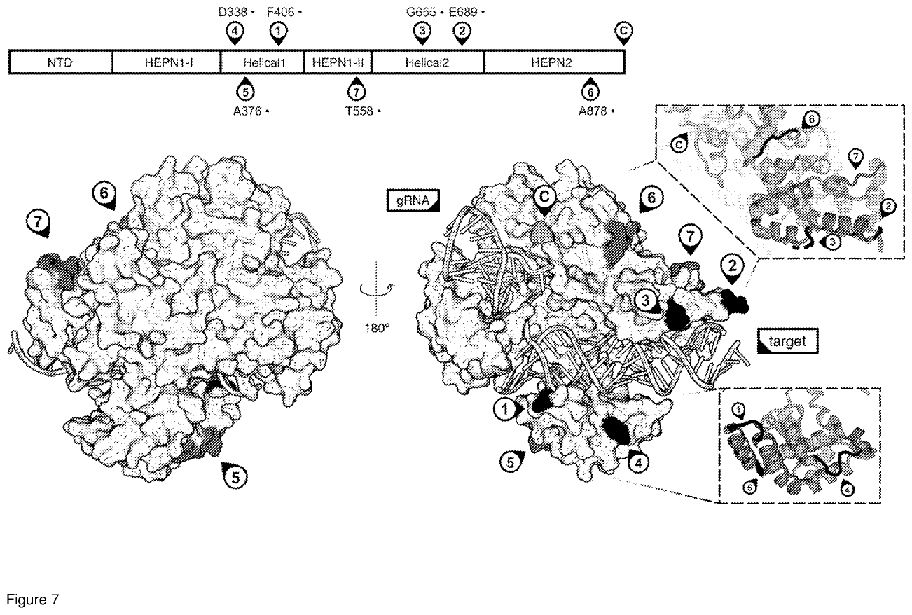

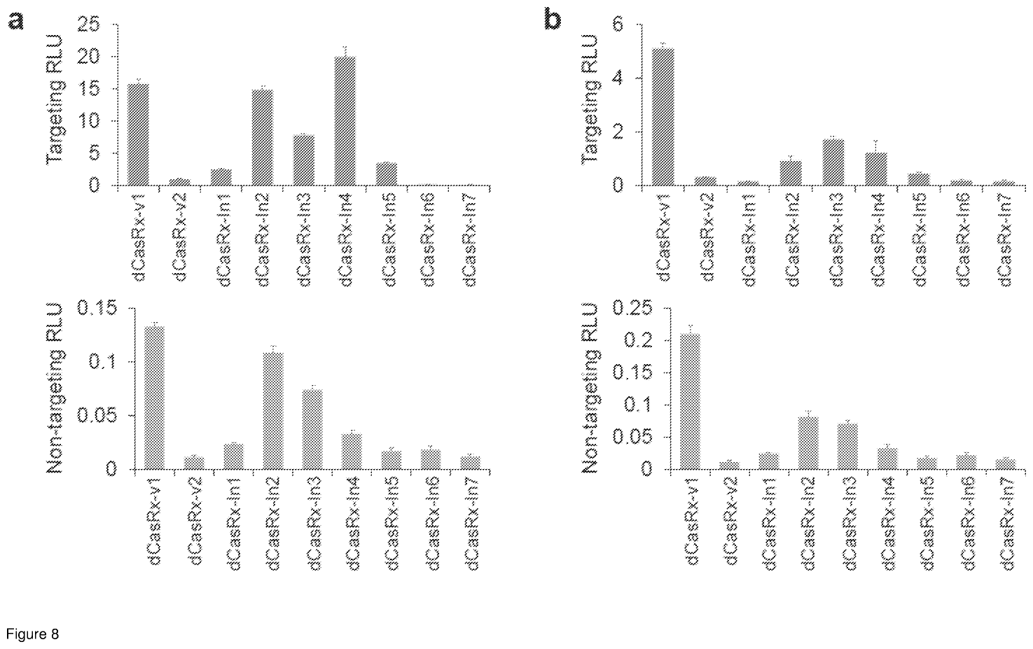

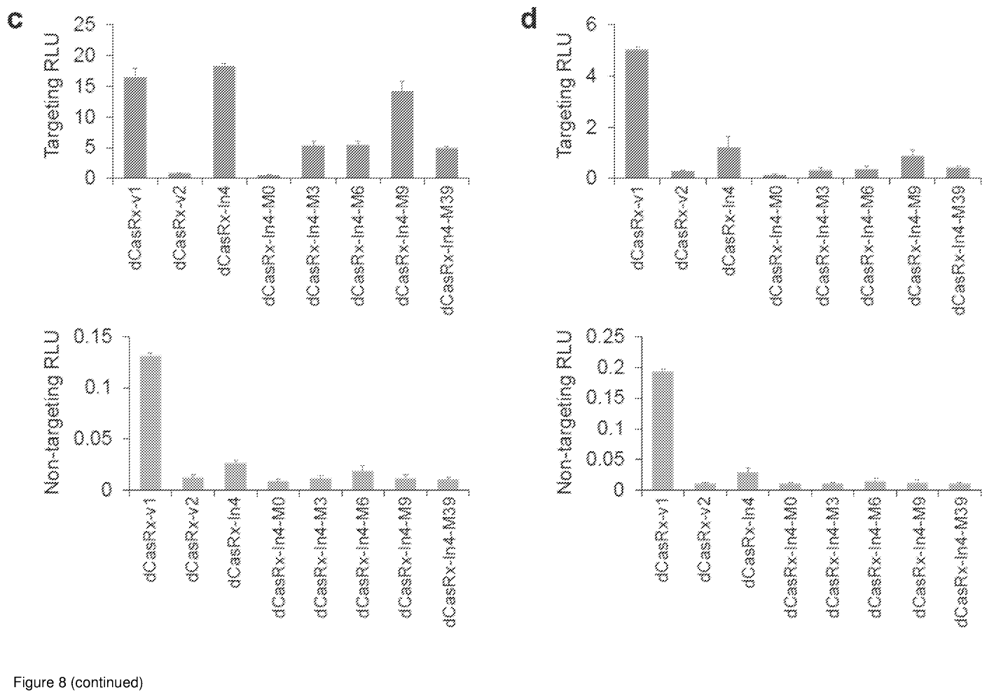

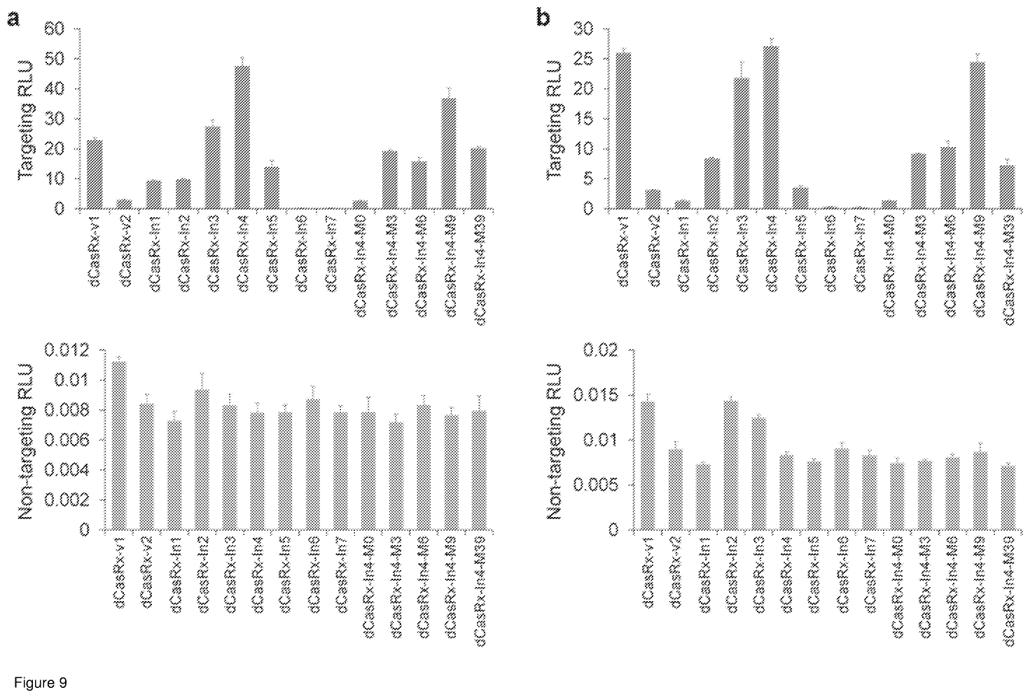

Thirdly, the inventors discovered that if ADAR2 is fused at the C-terminus, it can have too much flexibility to act on off-target sites. Hence, the Cas13 structure was examined to identify some internal sites where the ADAR2 can be linked to (see FIG. 7). Four of the identified sites are located closer to the guide RNA-target RNA duplex than the C-terminus. It was found that fusion of ADAR2 to most of these internal sites resulted in similar on-target efficiencies but lower off-target editing than the original C-terminus fusion construct. The usage of a mutant ADAR2 domain at an internal site effectively reduces off-target editing to background levels in proof-of-concept luciferase recovery assays (see FIGS. 8 and 9).

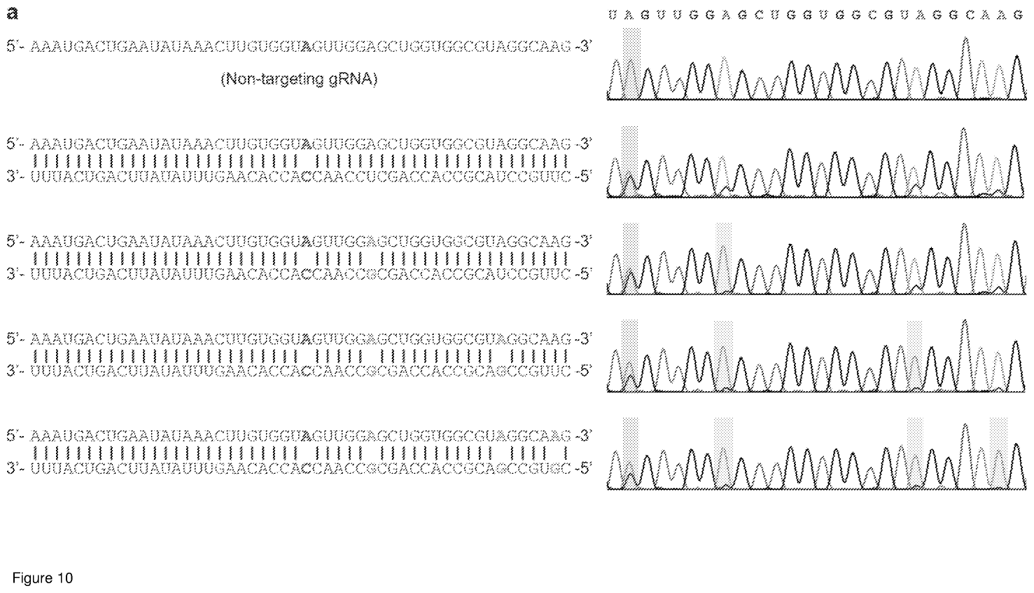

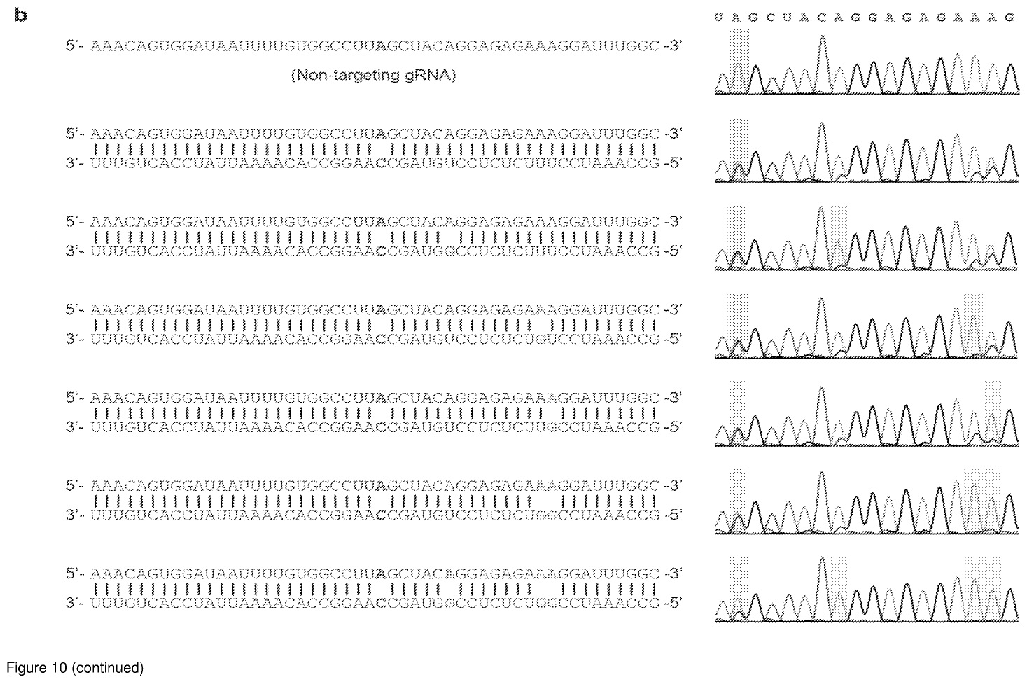

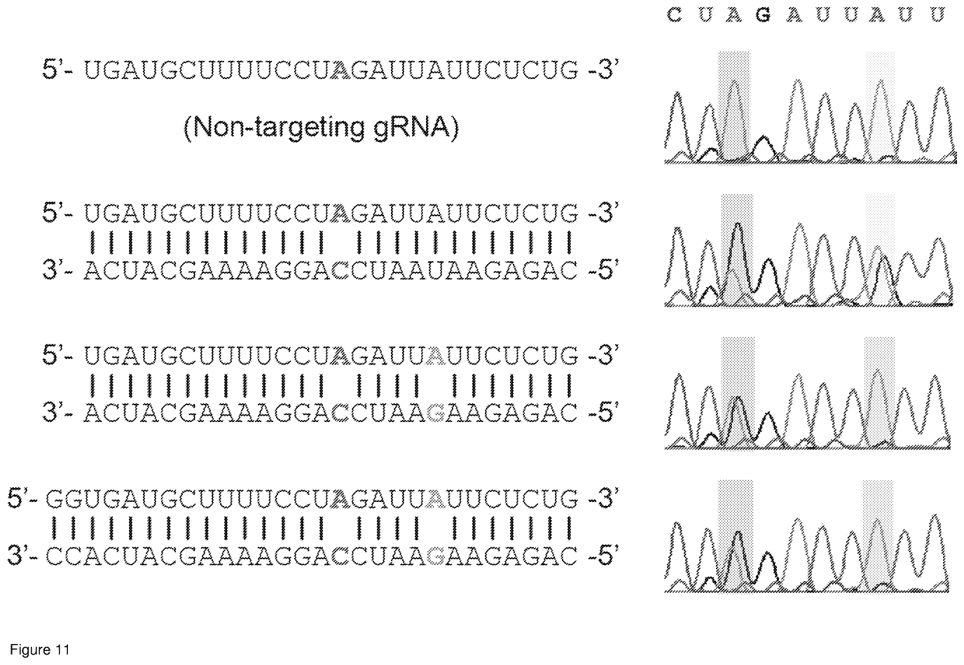

Fourthly, the inventors observed that there were occasionally some cis off-targets located at the guide RNA-target RNA interface. As it was found that a guanosine opposite an adenosine (an A-G mismatch) is highly unfavorable for editing by ADAR, i.e. more unfavorable than the standard A-U match, these cis off-targets could be eliminated by putting guanosines opposite adenosines that were wrongly edited in the guide RNA. This strategy was found to work for both dCas13b-ADAR2dd (see FIG. 10) and dCasRx-ADAR2dd (see FIG. 11).

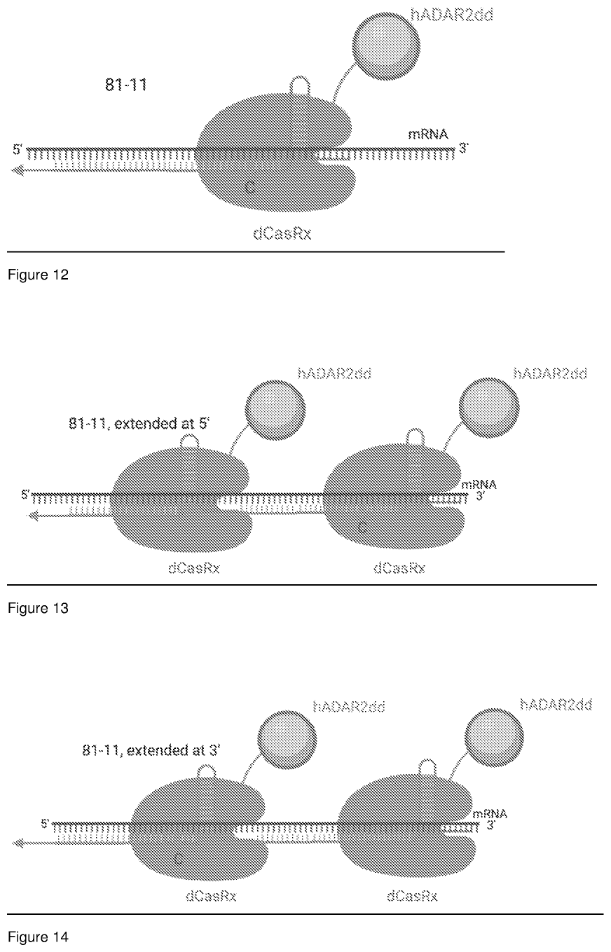

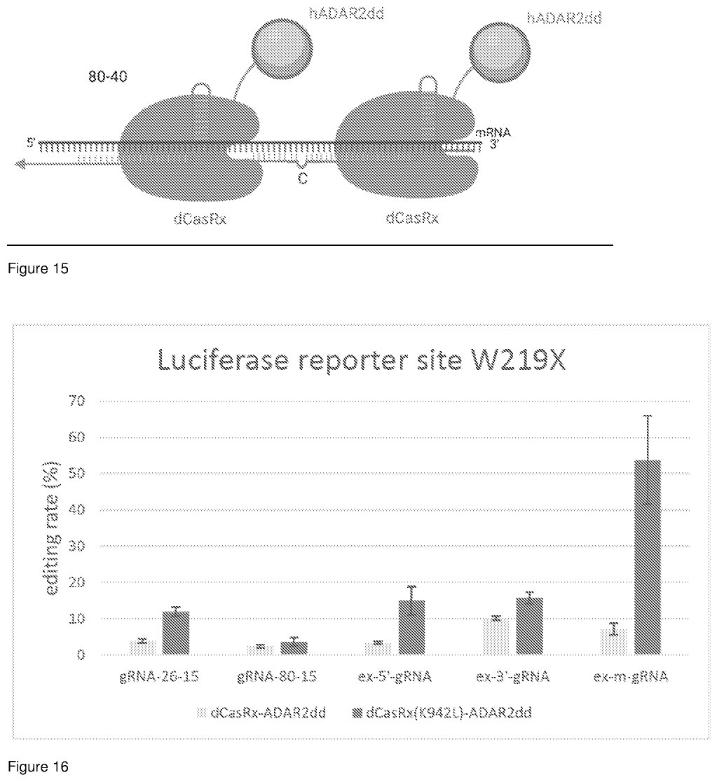

Based on these findings, an optimized construct, referred to as “xPERT” (CasRx-based programmable editing of RNA technology), was produced that comprises dCasRx fused internally after D338 to a human ADAR2 deaminase domain with the mutation H460D (H145D using the positional number of SEQ ID NO:1). Said xPERT platform, which consist of a dCasRx linked with either a wildtype or a rationally engineered ADAR2 deaminase domain, could precisely target and edit RNA with a 26 bp gRNA. However, it was found that this system could not edit some sites very well, which might be due to chromatin accessibility, sequence complexity, or hindrance from other RNA-binding proteins (RBPs). Therefore, an extended gRNA system was created to improve editing in these difficult sites. The gRNA was extended in several different ways. Firstly, only the spacer length was extended (FIG. 12). Secondly, two gRNAs were joined together. The intended target site is the adenosine with an adjacent ‘C’ bulge in the bound gRNA. The gRNA could be extended either upstream or downstream of the target site (FIGS. 13 and 14). The hypothesis was that the second dCasRx-ADAR2dd complex could help the first one to access the targeted editing site by displacing other RBPs or smoothing out the RNA secondary structure. Finally, because ADARs tend to bind in the center of long double strand RNA, it was also tried to change the mismatch to the middle of the two gRNAs (FIG. 15).

CasRx could however process its own gRNA, which will cause the extended gRNA to be cleaved and separated. Lys942 of dCasRx was shown to be critical for this process. Lys942 of dCasRx was thus mutated to Leu to abolish pre-crRNA cleavage (Konermann et al. (2018) Cell 173(3), 665-676). Said K942L mutation is herein also referred to as 940L, using the positional numbering of SEQ ID NO:2.

A luciferase reporter assay was used to check the editing efficiency. A nonsense mutation of W219X in the luciferase reporter was introduced, and with A-to-I editing, it will recover the luciferase signal. It was found that the K942L mutation could improve the editing efficiency in this site, and K942L with extended gRNA in 5′ or 3′ further increased the editing level to 3.8 to 4.0-fold, compared with dCasRx paired with normal gRNA. When the mismatch was set in the middle, the editing level was increased to more than 50%, 14.3-fold to dCasRx paired with normal gRNA (FIG. 16).

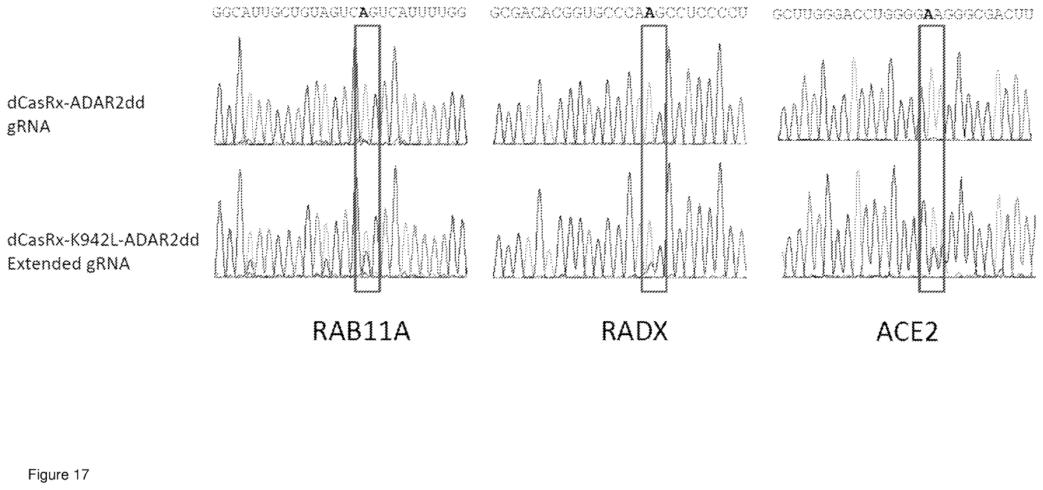

Next other difficult-to-edit sites in other genes (when using the dCasRx system) were tested and their editing levels checked. It was found that dCasRx K942L with extended gRNA could increase the editing level greatly in most of these sites (FIG. 17).

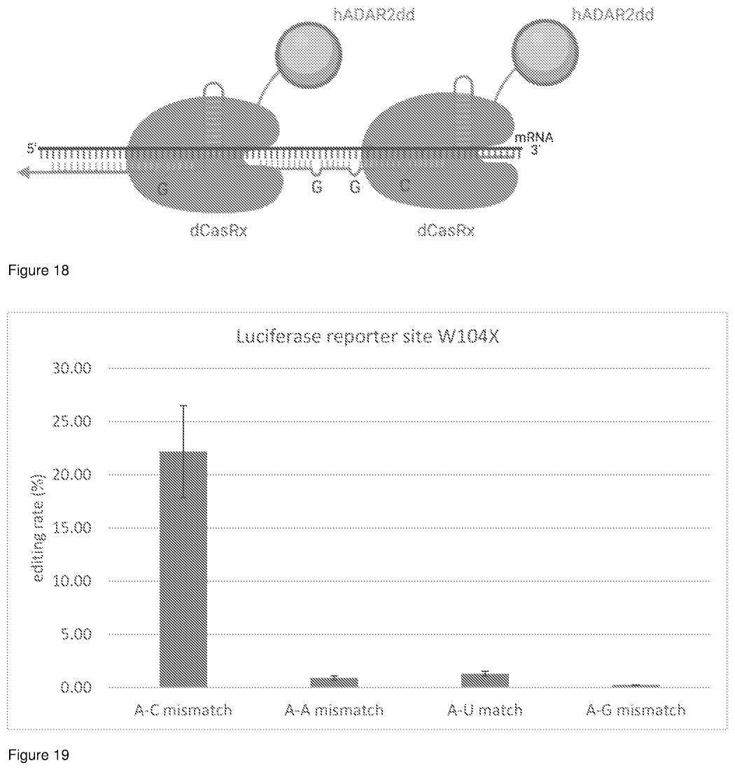

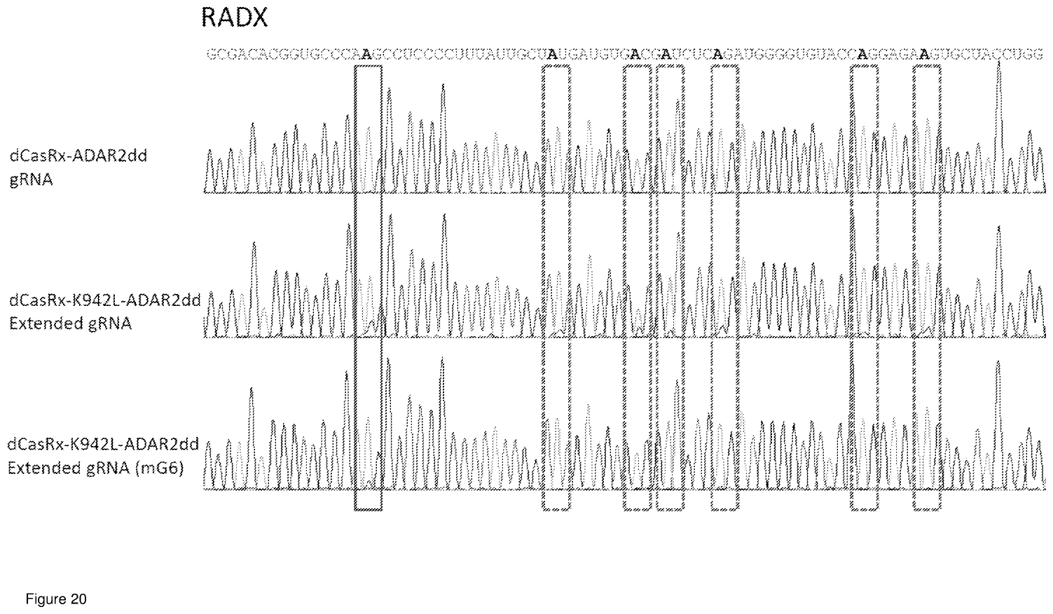

When extended gRNA was used, it was found that it will sometimes cause cis off-target editing, especially in the middle of gRNA match region. An A-G mismatch was known to suppress deamination of ADAR2, therefore a G mismatch in the gRNAs was created to reduce cis off-target editing (FIG. 18). It was found that an A-G mismatch could decrease the editing level dramatically in luciferase reporter assay (FIG. 19). Next, it was tried to remove the cis off-target editing in RADX gene induced by extended gRNA. Therefore, a new extended gRNA including guanosines opposite potential off-target adenosines was designed (extended gRNA mG6). It was found that extended gRNA mG6 could remove most of the off-targeting, but it may also decrease the on-target editing level (FIG. 20).

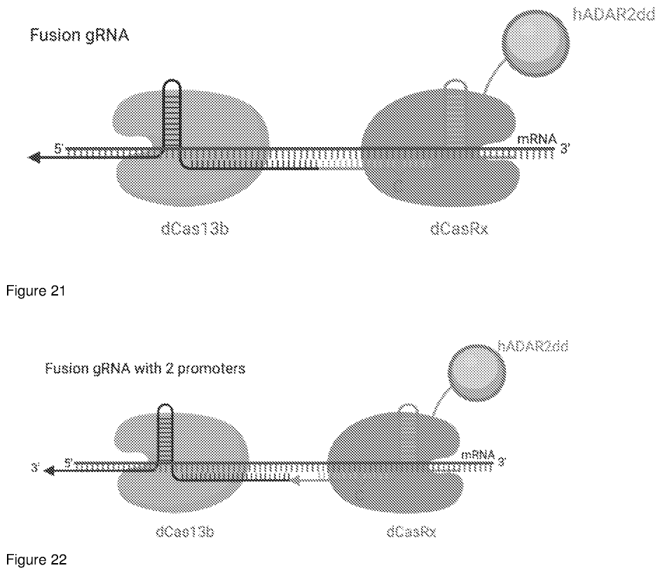

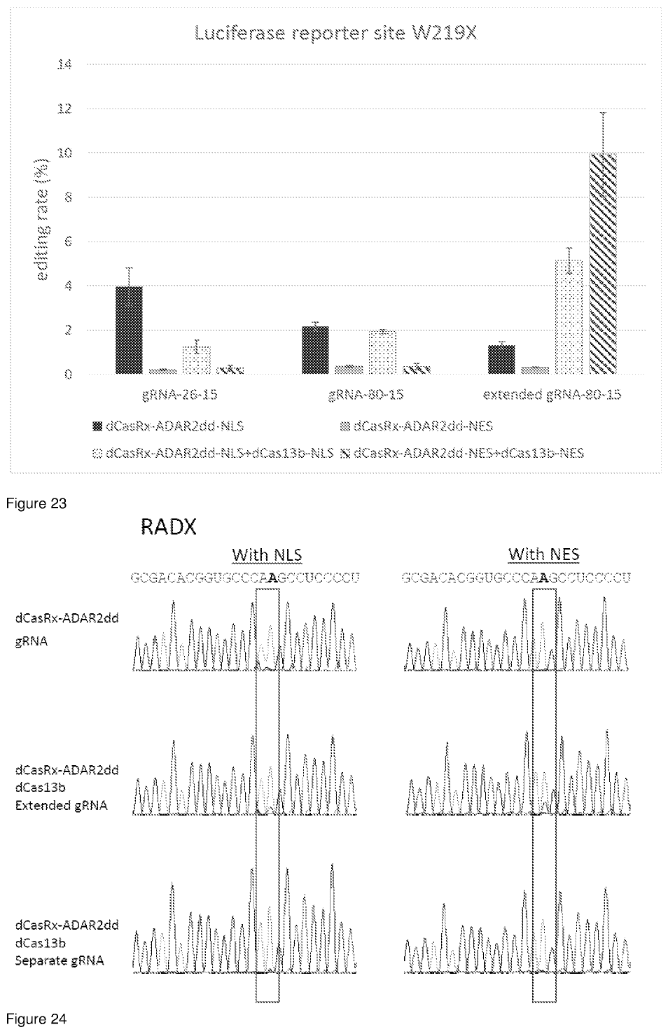

Another version of “extended gRNA” was thus designed. Here, two individual gRNAs were fused together. The first one is the same gRNA that recruits the dCasRx-ADAR2dd enzyme for programmable RNA editing. The second gRNA has a different stem loop and will recruit dCas13b to bind to an adjacent site. Different Cas proteins may possess different targeting features, so dCas13b can help dCasRx to edit some regions that it cannot bind. Furthermore, as there is only one ADAR2 deaminase domain linked with dCasRx in this system, it can create less off-targeting. The fusion extended gRNA was expressed under a single U6 promoter (FIG. 21). As a comparison, the dCas13b gRNA and the dCasRx gRNAs were also expressed using two different U6 promoters (FIG. 22). It was found that the extended gRNA using dCas13b could help dCasRx to edit some sites in the luciferase reporter system and in endogenous genes. Interestingly, whereas dCasRx itself cannot work very well with NES, with the help of dCas13b, this system could edit efficiently in the cytoplasm. Using two U6 promoters to express two gRNA separately for dCasRx and dCas13b could not increase editing, suggesting that a physical linkage between the two gRNA is important (FIGS. 23 and 24).

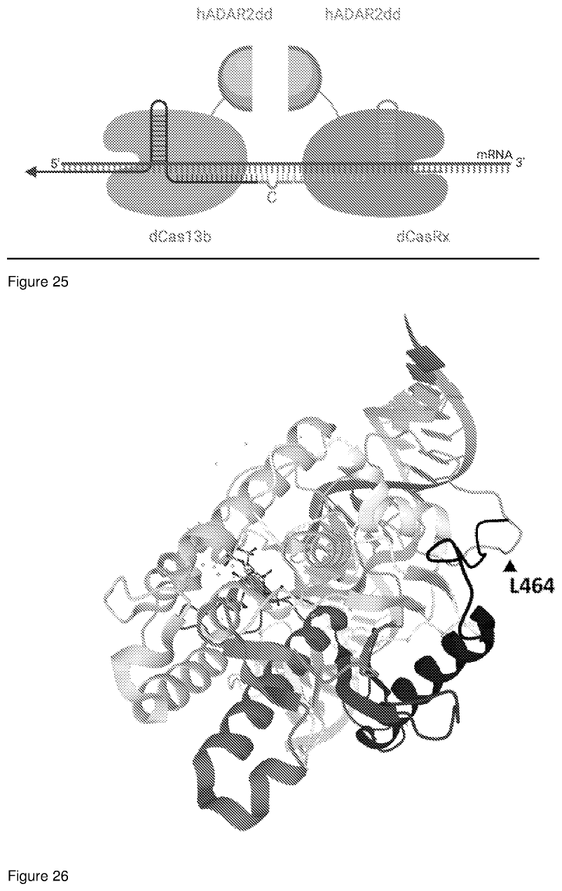

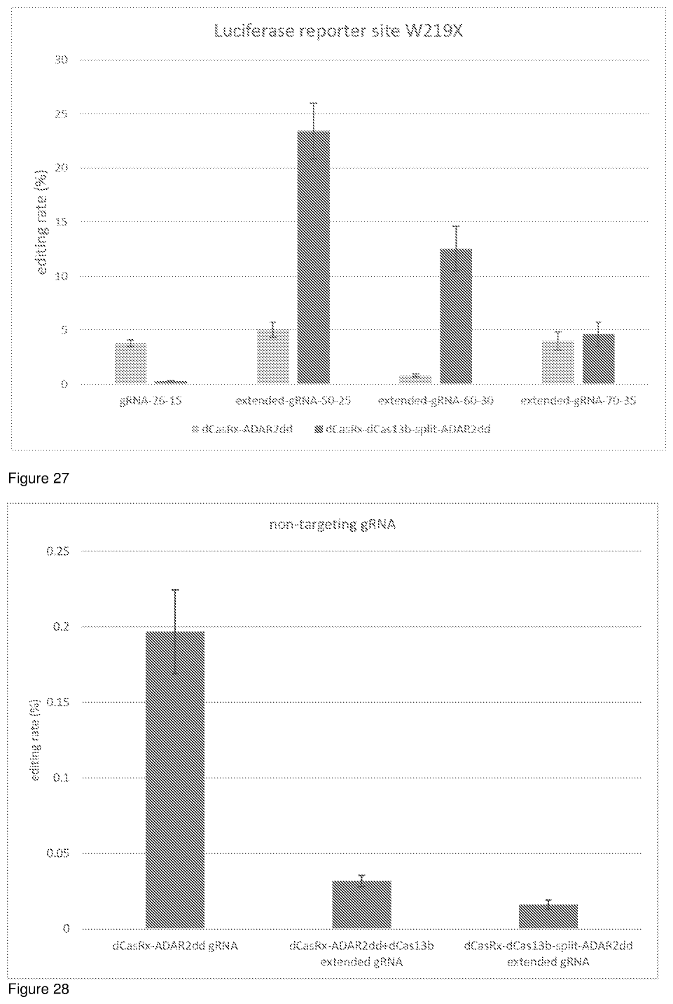

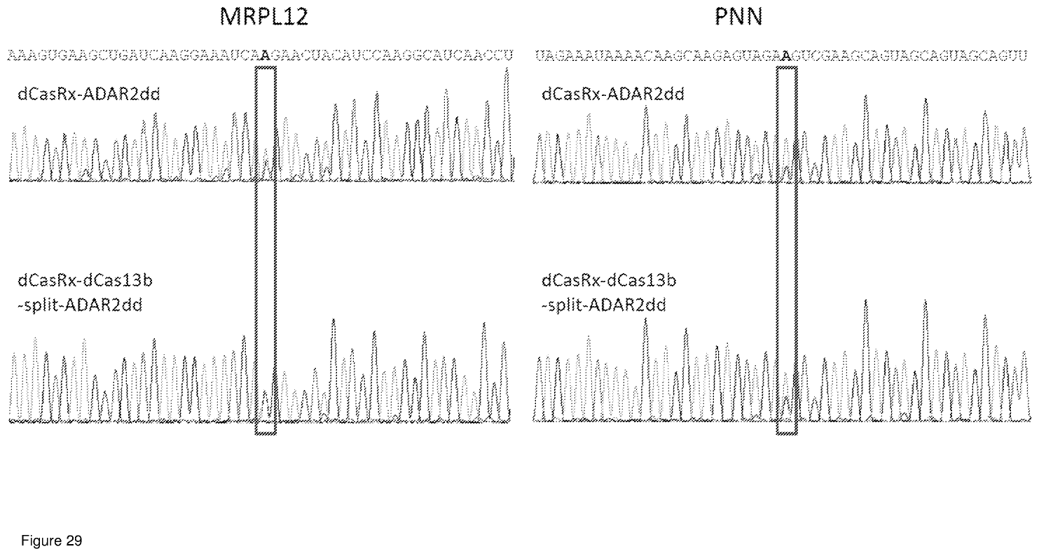

Overexpression of ADAR2 deaminase domain will cause off-target editing in the whole transcriptome. To further decrease the off-target in extended gRNA system, the ADAR2 deaminase domain was split to two parts, and the parts fused to the N-terminal of dCasRx and C-terminal of dCas13b, respectively (FIG. 25). In theory, this system can be active only when dCasRx and dCas13b are in close proximity. The hADAR2 deaminase domain is split at L464 (L149 using the positional numbering of SEQ ID NO:1) in a flexible region (FIG. 26). The split ADAR system utilizes an extended gRNA with the mismatch distance in the middle. In luciferase reporter assays, it was found that the split ADAR2dd system could create editing in targeting sites, with very low off-targeting efficiency as compared with other dCasRx-ADAR2dd systems (FIGS. 27 and 28). The split ADAR2dd system was also tested in endogenous sites, and it was found that it has comparable or higher editing efficiency with normal dCasRx-ADAR2dd (FIG. 29).

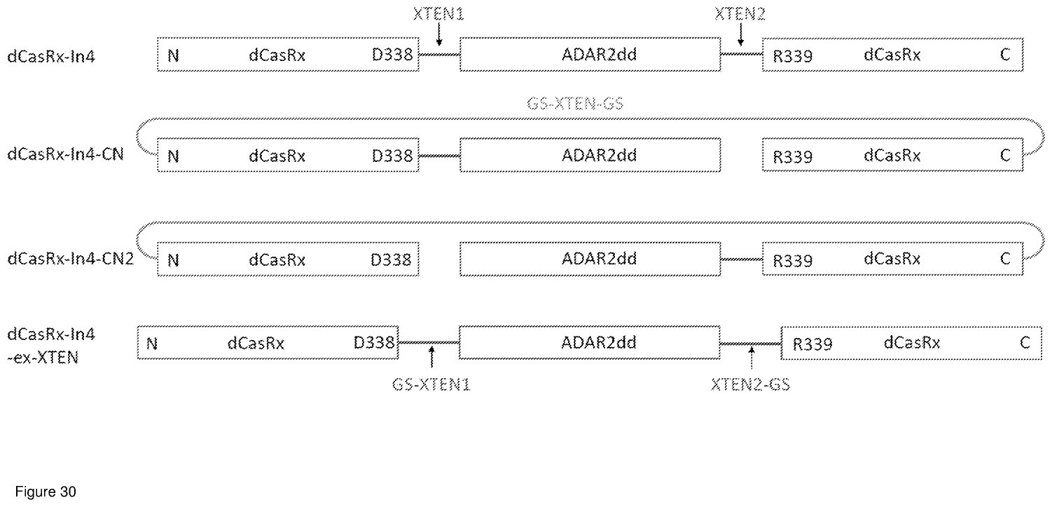

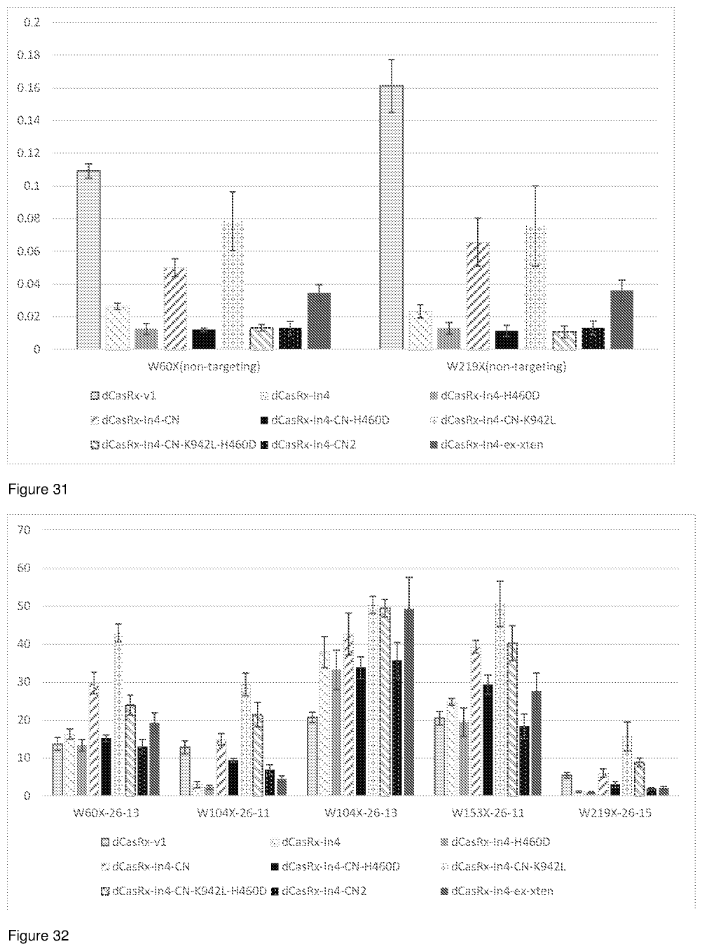

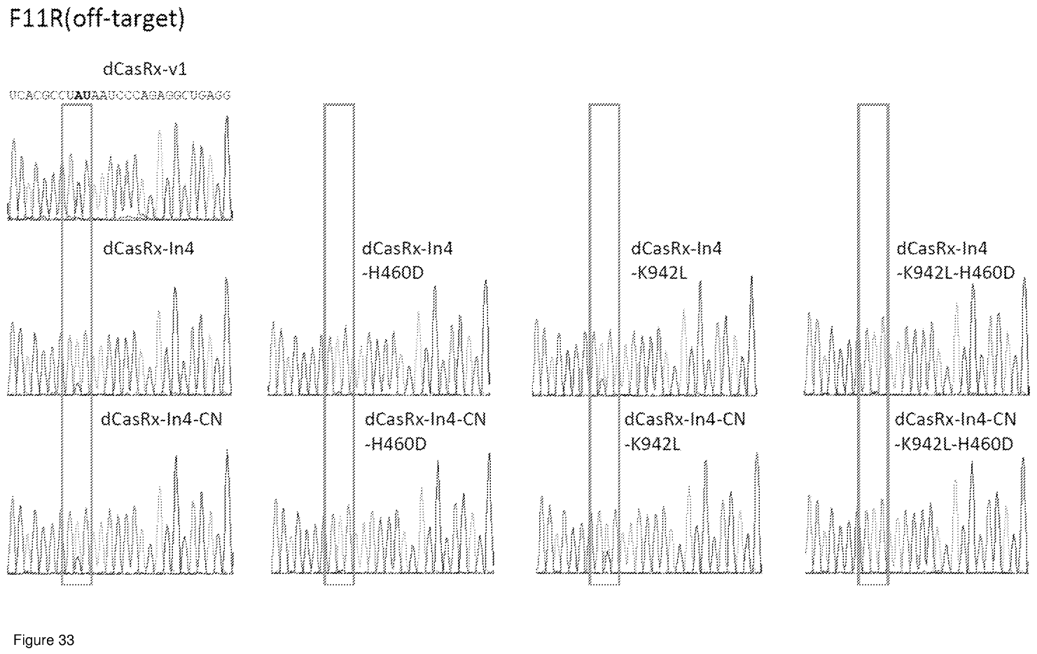

The inventors noticed from the crystal structure of ADAR2dd that its N- and C-terminus are relatively far apart. However, when ADAR2dd is fused at an internal site of dCasRx, the N- and C-terminus of the deaminase are forced to come closer together, which may strain the domain. Hence, to free ADAR2dd from this strain, the inventors rearranged the fragments by moving the back portion of dCasRx to the front (FIG. 30). It was observed from luciferase reporter assays that the newly rearranged construct exhibited higher on-target activity and further introduction of the K942L mutation increased the activity even more (FIGS. 31 and 32). In addition, introduction of the H460D mutation into the rearranged constructs improved their specificity (FIGS. 31 and 32), which was verified by assessing off-target editing of an endogenous gene, F11R (FIG. 33).

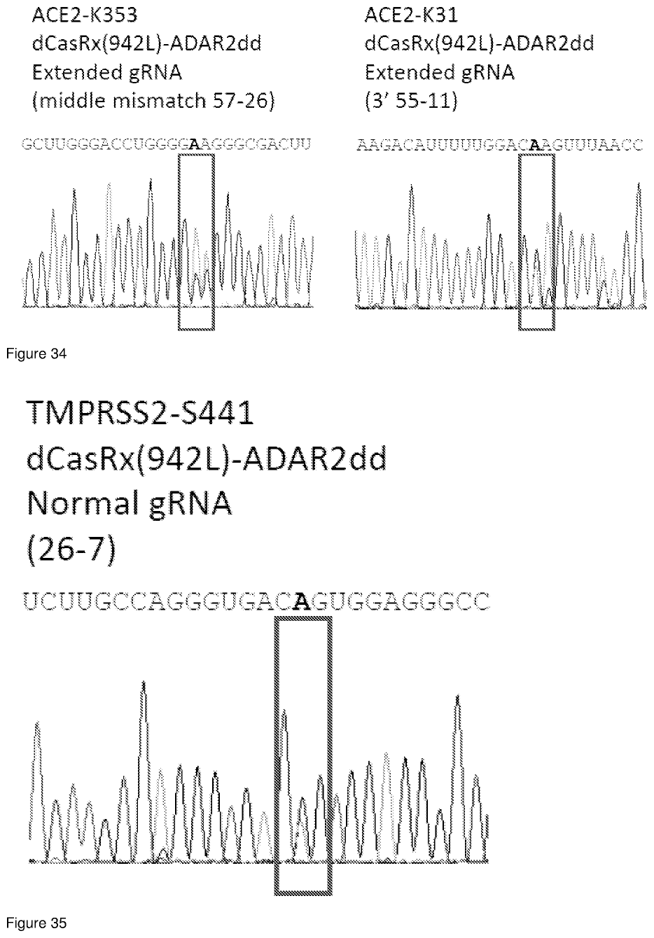

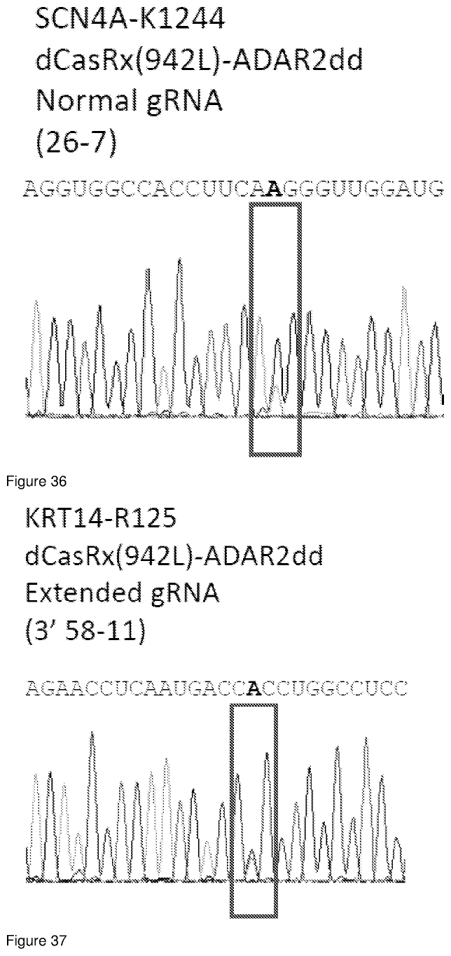

To demonstrate the utility of the xPERT platform, the inventors applied their technology to clinically relevant genes. It was found that specific sites within the ACE2 (FIG. 34), TMPRSS2 (FIG. 35), SCN4A (FIG. 36), and KRT14 (FIG. 37) genes could be robustly edited.

In summary the inventors discovered multiple options to further improve an ADAR-base RNA editing technology by various modifications to the active ADAR moiety, the Cas family targeting moiety and the gRNA.

Based on the above findings, the invention, in a first aspect, covers an isolated polypeptide comprising or consisting of
(1) a first polypeptide domain comprising an amino acid sequence that
   (i) shares at least 70, preferably at least 80, more preferably at least 90%, most preferably at least 95% sequence identity with the amino acid sequence set forth in SEQ ID NO:1 over its entire length; and
   (ii) comprises the amino acid substitution 1730 using the positional numbering of SEQ ID NO:1; and, optionally,
   (iii) comprises amino acid substitutions at any one or more of the positions corresponding to positions 145, 33, 34, 36, 139, 140, 142, 143, 154, 155, 156, 158, 159, 160, 162, and 164 of SEQ ID NO:1 (hADAR2dd);
(2) a second polypeptide domain comprising an amino acid sequence that shares at least 70, preferably at least 80, more preferably at least 90%, most preferably at least 95% sequence identity with
   (i) the amino acid sequence set forth in SEQ ID NO:2 over its entire length (dCasRx) and comprises the amino acid substitutions 239A, 244A, 858A, and 863A using the positional numbering of SEQ ID NO:2; or
   (ii) the amino acid sequence set forth in SEQ ID NO:3 over its entire length (dCas13b) and comprises the amino acid substitutions 133A and 1058A, using the positional numbering of SEQ ID NO:3;
wherein the first polypeptide domain is fused to the second polypeptide domain or inserted into the second polypeptide domain;
with the proviso that if the second polypeptide domain comprises an amino acid sequence that shares at least 70, preferably at least 80, more preferably at least 90%, most preferably at least 95% sequence identity with the amino acid sequence set forth in SEQ ID NO:3 and comprises the amino acid substitutions 133A and 1058A, using the positional numbering of SEQ ID NO:3, the first polypeptide domain does not have the amino acid sequence of SEQ ID NO:1 with the amino acid substitution 173Q in combination with one of 33E, 36L, 140G/S/E, 158D, 159E, 160Q, and 162E.

The isolated polypeptides have RNA deaminase activity in isolated form as they comprise the first polypeptide domain having sufficient structural similarity to human ADAR2. This means that they can convert a target A in an RNA molecule to I and thus introduce a A-to-G conversion. In various embodiments, these first polypeptide domains comprise, consist essentially of or consist of the amino acid sequence as set forth in SEQ ID NO:1 including the given mutations, with the 1730 mutation providing for increased enzymatic activity and any one or more of the mutations in positions 145, 33, 34, 36, 139, 140, 142, 143, 154, 155, 156, 158, 159, 160, 162, and 164 of SEQ ID NO:1 providing for less off-target activity on generic RNA. The polypeptide consisting of the amino acid sequence set forth in SEQ ID NO:1 is also referred to as “hADAR2dd” or “ADAR2” herein.

The isolated polypeptides also have RNA targeting activity in isolated form as they comprise the second polypeptide domain having sufficient structural similarity to a member of the Cas family of endonucleases, in particular CasRx (SEQ ID NO:2) or Cas13b (SEQ ID NO:3). In various embodiments, these first polypeptide domains comprise, consist essentially of or consist of the amino acid sequence as set forth in SEQ ID NO:2 or SEQ ID NO:3 including the given mutations. The polypeptides consisting of the amino acid sequences set forth in SEQ ID NO:2 and SEQ ID NO:3 are also referred to as “dCasRx” and “dCas13b”, respectively.

“Isolated”, as used herein, relates to the polypeptide in a form where it has been at least partially separated from other cellular components it may naturally occur or associate with. The polypeptide may be a recombinant polypeptide, i.e. polypeptide produced in a genetically engineered organism that does not naturally produce said polypeptide.

“Polypeptide”, as used herein, relates to polymers made from amino acids connected by peptide bonds. The polypeptides, as defined herein, can comprise 100 or more amino acids, preferably 200 or more amino acids. “Peptides”, as used herein, relates to polymers made from amino acids connected by peptide bonds. The peptides, as defined herein, can comprise 2 or more amino acids, preferably 5 or more amino acids, more preferably 10 or more amino acids, for example 10 to less than 100 amino acids.

The isolated polypeptides do, in case the second polypeptide domain is based on SEQ ID NO:3 (Cas13b) and comprises the inactivating mutations 133A and 1058A, specifically if the second polypeptide domain is with the exception of the two mutated sites 100% identical in length and sequence to SEQ ID NO:3, not comprise a first polypeptide domain that comprises only the mutation 173Q or the mutation 173Q in combination with (i) 33E, (ii) 36L, (iii) 140G/S/E, (iv) 158D, (v) 159E, (vi) 1600, or (vii) 162E. This limitation does not apply if the first polypeptide domain comprises 3 or more of the mutations listed above or any of the other mutation(s) recited herein alone or in combination with any one or more of 1730, 33E, 36L, 140G/S/E, 158D, 159E, 1600, and 162E. This limitation does also not apply if the second polypeptide domain is based on SEQ ID NO:2, as defined above.

In various embodiments of the isolated polypeptides, the first polypeptide domain comprises an amino acid substitution at the position corresponding to position 145 of SEQ ID NO:1.

In the first polypeptide domain, the recited positions may be mutated to any amino acid residue, such as G, A, V, L, I, F, M, C, S, T, D, E, N, Q, Y, W, R, K, H, and P, with the exception of the residue naturally occurring at this position. In the wildtype sequence, the respective positions are occupied by the following amino acid residues R33, R34, V36, A139, R140, F142, S143, H145, D154, R155, H156, N158, R159, K160, R162, 0164, and E173. Generally, it can be preferred that the target amino acid the respective residue is mutated to, is not a positively charged amino acid, i.e. is not R, K or H. In various embodiments, the target amino acid is thus chosen from G, A, V, L, I, F, M, C, S, T, D, E, N, Q, Y, W, and P. In various embodiments, the substitutions are selected from the following list of amino acid substitutions: 33G, 33A, 33E, 34G, 36L, 139C, 140A, 140D, 142Y, 143A, 145A, 145D, 154A, 155A, 155D, 156A, 158G, 158L, 159A, 159D, 160A, 160D, 160E, 160L, 162A, 164L, and 164V, using the positional numbering of SEQ ID NO:1.

When referring to amino acid substitutions, the known convention for their designation is used. “R33” thus means that the starting amino acid is R (Arg, arginine) in position 33, i.e. the letter in front of the number indicates the starting amino acid. If no such letter is given, the starting amino acid is not known or irrelevant. In turn, “33G” means that the residue in position 33 is mutated into G (Gly, glycine), i.e. the letter behind the number indicates the target amino acid. “R33G” thus indicates that the starting amino acid R in position 33 is mutated to G. If there are more than one option for the target amino acid, individual target amino acids by be separated by “/”, i.e. “33G/A/E”. This means that the residue in position 33 can be mutated into either of G, A and E. All amino acid residues are generally referred to herein by reference to their one letter code and, in some instances, their three-letter code. This nomenclature is well known to those skilled in the art and used herein as understood in the field.

In various embodiments of the isolated polypeptide, the first polypeptide domain at least comprises the amino acid substitution 145D using the positional numbering of SEQ ID NO:1. Said mutation may be accompanied by further mutations from the above list, but may also be used alone (i.e. only in combination with the 1730 mutation which is present in all embodiments). Preferred mutations and combinations of mutations are listed in the following Table (Table 1).

TABLE 1
ADAR2 mutations

| ADAR2 Mutant Label | Mutations |
|---|---|
| M0 | E173Q + V36L |
| M1 | E173Q + R33G |
| M2 | E173Q + R34G |
| M3 | E173Q + A139C |
| M4 | E173Q + R140A |
| M5 | E173Q + R150D |
| M6 | E173Q + F142Y |
| M7 | E173Q + S143A |
| M8 | E173Q + H145A |
| M9 | E173Q + H145D |
| M10 | E173Q + D154A |
| M11 | E173Q + R155A |
| M12 | E173Q + R155D |
| M13 | E173Q + H156A |
| M14 | E173Q + N158G |
| M15 | E173Q + N158L |
| M16 | E173Q + R159A |
| M17 | E173Q + R159D |
| M18 | E173Q + K160A |
| M19 | E173Q + K160D |
| M20 | E173Q + K160E |
| M21 | E173Q + K160L |
| M22 | E173Q + R162A |
| M23 | E173Q + Q164L |
| M24 | E173Q + Q164V |
| M25 | E173Q + R33A + V36L |
| M26 | E173Q + R33E + V36L |
| M27 | E173Q + R34G + K160D |
| M28 | E173Q + R140A + S143A |
| M29 | E173Q + R140G + S143A |
| M30 | E173Q + H145D + R155A |
| M31 | E173Q + H145D + R159D |
| M32 | E173Q + H145D + K160D |
| M33 | E173Q + R155A + N158G |
| M34 | E173Q + R155A + K160D |
| M35 | E173Q + N158G + K160A |
| M36 | E173Q + N158G + K160L |
| M37 | E173Q + N158G + Q164L |
| M38 | E173Q + R159D + K160A |
| M39 | E173Q + K160D + Q164L |
| M40 | E173Q + R140A + S143A + K160D |
| M41 | E173Q + H145D + R155A + N158G |
| M42 | E173Q + H145D + N158G + K160L |
| M43 | E173Q + R470A + N158G + K160A |
| M44 | E173Q + N158G + R159D + K160A |
| M45 | E173Q + R155A + N158G + R159D + K160A |

In various embodiments, the polypeptide of the invention comprises a first polypeptide domain that comprises or consists of an amino acid sequence that is at least 60%, 65%, 70%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93% Y, 93.5%, 94%, 94.5%, 95%, 95.5%, 96%, 96.5%, 97%, 97.5%, 98%, 98.5%, 99%, 99.1%, 99.2%, 99.3%, 99.4%, 99.5%, 99.6%, or 99.7% identical or homologous to the amino acid sequence set forth in SEQ ID NO:1 over its entire length. This sequence identity/homology relates to the complete sequence of the first polypeptide domain including any one or more of the given mutations. In various embodiments, the first polypeptide domain does not comprise any mutation or sequence variation outside the positions indicated herein, i.e. is 100% identical to the sequence set forth in SEQ ID NO:1 (over its entire length) with the exception of positions 173 and any one or more of 145, 33, 34, 36, 139, 140, 142, 143, 154, 155, 156, 158, 159, 160, 162, and 164. In various embodiments, it can be preferred that the first polypeptide domain does comprise the 1730 mutation and 1, 2, 3, 4, 5 or 6, for example 1, 2, 3, 4 or 5, preferably 1, 2, 3 or 4, more preferably 1, 2 or 3, additional mutations in any of the listed positions. In various embodiments, at least the mutations 1730 and 145D are present. In any of the foregoing embodiments, the first polypeptide domain may also comprise N- and/or C-terminal truncations relative to SEQ ID NO:1, i.e. may lack 1 to 30 amino acids from either or both of its termini. It is preferred that such truncations do not impair its activity. In case truncated versions of SEQ ID NO:1 are comprised in the polypeptides of the invention, it is preferred that the remaining sequence shares the sequence identity/homology disclosed above, preferably that the sequence identity with the exception of the mutated positions is 100%.

In various embodiments, the invention also features the first polypeptide domains disclosed herein, in particular those comprising any one or more of the above substitutions, as such, i.e. without the second polypeptide domain. In such embodiments, the isolated polypeptide of the invention comprises only the first polypeptide domain as defined herein, but not the second polypeptide domain.

The identity of nucleic acid sequences or amino acid sequences is generally determined by means of a sequence comparison. This sequence comparison is based on the BLAST algorithm that is established in the existing art and commonly used (cf. for example Altschul et al. (1990) “Basic local alignment search tool”, J. Mol. Biol. 215:403-410, and Altschul et al. (1997): “Gapped BLAST and PSI-BLAST: a new generation of protein database search programs”; Nucleic Acids Res., 25, p. 3389-3402) and is effected in principle by mutually associating similar successions of nucleotides or amino acids in the nucleic acid sequences and amino acid sequences, respectively. A tabular association of the relevant positions is referred to as an “alignment.” Sequence comparisons (alignments), in particular multiple sequence comparisons, are commonly prepared using computer programs which are available and known to those skilled in the art.

A comparison of this kind also allows a statement as to the similarity to one another of the sequences that are being compared. This is usually indicated as a percentage identity, i.e. the proportion of identical nucleotides or amino acid residues at the same positions or at positions corresponding to one another in an alignment. The more broadly construed term “homology”, in the context of amino acid sequences, also incorporates consideration of the conserved amino acid exchanges, i.e. amino acids having a similar chemical activity, since these usually perform similar chemical activities within the protein. The similarity of the compared sequences can therefore also be indicated as a “percentage homology” or “percentage similarity.” Indications of identity and/or homology can be encountered over entire polypeptides or genes, or only over individual regions. Homologous and identical regions of various nucleic acid sequences or amino acid sequences are therefore defined by way of matches in the sequences. Such regions often exhibit identical functions. They can be small, and can encompass only a few nucleotides or amino acids. Small regions of this kind often perform functions that are essential to the overall activity of the protein. It may therefore be useful to refer sequence matches only to individual, and optionally small, regions. Unless otherwise indicated, however, indications of identity and homology herein refer to the full length of the respectively indicated nucleic acid sequence or amino acid sequence.

In various embodiments of the isolated polypeptides, the first polypeptide domain has the amino acid sequence set forth in any one of SEQ ID NOS:4-49 or is a variant thereof that has a sequence identity of at least 95%, at least 96%, at least 97%, at least 98%, or at least 99%, including truncated variants, with the mutated positions being invariable.

In various embodiments, the isolated polypeptide comprises a second polypeptide domain according to (2)(i) that comprises an amino acid substitution in the position corresponding to position 940 of SEQ ID NO:2, preferably 940L.

In various embodiments, the polypeptide of the invention comprises a second polypeptide domain that comprises or consists of an amino acid sequence that is at least 60%, 65%, 70%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.6%, 98.7%, 98.8%, 98.9%, 99.0%, 99.1%, 99.2%, 99.3%, 99.4%, 99.5%, 99.6%, 99.7%, or 99.8% identical or homologous to the amino acid sequence set forth in SEQ ID NO:2 or SEQ ID NO:3 over its entire length. This sequence identity/homology relates to the complete sequence of the second polypeptide domain including any one or more of the given mutations. In various embodiments, the second polypeptide domain does not comprise any mutation or sequence variation outside the positions indicated herein, i.e. is 100% identical to the sequence set forth in SEQ ID NO:2 (over its entire length) with the exception of positions 239, 244, 858, 863 (239A, 244A, 858A, and 863A) and optionally 940, using the positional numbering of SEQ ID NO:2 or is 100% identical to the sequence set forth in SEQ ID NO:3 (over its entire length) with the exception of positions 133 and 1058 (133A and 1058A) using the positional numbering of SEQ ID NO:3. In any of the foregoing embodiments, the second polypeptide domain may also comprise N- and/or C-terminal truncations relative to SEQ ID NO:2 or SEQ ID NO:3, i.e. may lack 1 to 30 amino acids from either or both of its termini. It is preferred that such truncations do not impair its activity. In case truncated versions of SEQ ID NO:2 or SEQ ID NO:3 are comprised in the polypeptides of the invention, it is preferred that the remaining sequence shares the sequence identity/homology disclosed above, preferably that the sequence identity with the exception of the mutated positions is 100%.

The isolated polypeptide of the invention may, in various embodiments, comprise a second polypeptide domain having the amino acid sequence set forth in any one of SEQ ID NOS:50 to 52.

The isolated polypeptides of the invention are fusion proteins in that the first and second polypeptide domain are fused to each other. This means that both form part of a polypeptide and are linked to each other either directly or via additional peptide sequence via a peptide bond. In various embodiments, the first polypeptide domain is located C-terminally to the second polypeptide domain. This may mean that the first polypeptide domain is fused to the C-terminus of the second polypeptide domain either directly or via a linker sequence. In such embodiments, the structure of the polypeptide of the invention is, in N to C-terminal orientation, thus:
PPD2-L-PPD1
   wherein PPD2 is the second polypeptide domain as defined herein, PPD1 is the first polypeptide domain as defined herein and L is a peptide bond or linker peptide sequence. Suitable linker peptide sequences are defined below.

In various other embodiments, the first polypeptide domain is inserted into the second polypeptide domain. “Inserted”, as used in this context, means that the full length sequence of the second polypeptide domain is split into two parts and that the first polypeptide domain is, in one embodiment, located between those such that its N-terminus is either directly or via a linker sequence linked to the C-terminus of the N-terminal part of the split second polypeptide domain and its C-terminus is either directly or via a linker sequence linked to the N-terminus of the C-terminal part of the split second polypeptide domain. In such embodiments, the structure of the polypeptide of the invention may be, in N- to C-terminal orientation:
PPD2.1-L-PPD1-L-PPD2.2
   wherein PPD2.1 is the N-terminal part of the second polypeptide domain as defined herein, PPD2.2 is the C-terminal part of the second polypeptide domain as defined herein, wherein PPD2.1 and PPD2.2 if directly fused to each other would form PPD2, PPD1 is the first polypeptide domain as defined herein, and L is a peptide bond or linker peptide sequence. If L is a linker, it may be the linker of SEQ ID NO:55, or the sequence GS plus SEQ ID NO:55, wherein GS is N-terminal to SEQ ID NO:55, in particular in the first L, or C-terminal to SEQ ID NO:55, in particular in the second L.

Alternatively, “inserted”, as used herein, also means that the first polypeptide domain is fused to one fragment of the split second polypeptide domain, i.e. its N-terminus is fused to the C-terminus of the N-terminal part of the second polypeptide domain or its C-terminus is fused to the N-terminus of the C-terminal part of the second polypeptide domain, and the N-terminal part of the second polypeptide domain is linked by its N-terminus to the C-terminus of the C-terminal part of the second polypeptide domain, either directly or via a linker. In such embodiments, the structure of the polypeptide of the invention may be, in N- to C-terminal orientation:
PPD2.2-L-PPD2.1-L-PPD1, or
PPD1-L-PPD2.2-L-PPD2.1
   wherein PPD2.1 is the N-terminal part of the second polypeptide domain as defined herein, PPD2.2 is the C-terminal part of the second polypeptide domain as defined herein, wherein PPD2.1 and PPD2.2 if directly fused to each other in form of PPD2.1-PPD2-2 would form PPD2, PPD1 is the first polypeptide domain as defined herein, and L is a peptide bond or linker peptide sequence. If L is a linker, it may be the linker of SEQ ID NO:55 or SEQ ID NO:55 flanked by two GS (Gly-Ser) sequences. In these constructs, the N- and C-terminus of PPD1 are farther apart than if inserted between two fragments of the PPD2. This puts less strain on the deaminase domain.

If the first polypeptide domain is inserted into the second polypeptide domain, the site for insertion or split of the second polypeptide domain is typically selected such that the two parts of the second polypeptide domain are still functional and preferably not impaired in their functionality relative to the intact domain. In various embodiments, the first polypeptide domain is thus inserted after position 338, 655 or 689 of the second polypeptide domain, using the positional numbering of SEQ ID NO:2. “Inserted after”, as used in this context, means that the first polypeptide domain is linked, either directly or via a linker, to the C-terminus of the amino acid in position 338, the N-terminus of the amino acid in position 339, or both. More specifically, the first polypeptide domain may
(A) with its N-terminus be linked to the C-terminus of the amino acid residue in position 338 of the second polypeptide, optionally via a linker, and (1) with its C-terminus to the N-terminus of the amino acid residue in position 339 of the second polypeptide domain, optionally via a linker, or (2) the C-terminus of the C-terminal part of the second polypeptide domain is linked to the N-terminus of the N-terminal part of the second polypeptide domain, optionally via a linker; or
(B) with its C-terminus be linked to the N-terminus of the amino acid residue in position 339 of the second polypeptide, optionally via a linker, and the C-terminus of the C-terminal part of the second polypeptide domain is linked to the N-terminus of the N-terminal part of the second polypeptide domain, optionally via a linker.

In the above embodiments, where the first polypeptide domain is inserted after position 338, 655 or 689 of the second polypeptide domain, the second polypeptide domain is preferably that according to (2)(i), i.e. is based on SEQ ID NO:2. A particularly preferred insertion site is after position 338 of the second polypeptide domain, using the positional numbering of SEQ ID NO:2, i.e. the linkage is to the residue in position 338, the residue in position 339 or both.

Accordingly, in various embodiments, “PPD2.1” as used herein, refers to the amino acid residues corresponding to amino acids 1-338 of SEQ ID NO:2 and “PPD2.2”, as used herein, refers to the amino acid residues corresponding to amino acids 339-967 of SEQ ID NO:2. In various embodiments, PPD2.2 includes the 940L mutation, using the positional numbering of SEQ ID NO:2.

Irrespective of whether the first polypeptide domain is fused to the terminus of the second polypeptide domain or inserted therein and whether the first or second polypeptide domains are split or not, the isolated polypeptides of the invention can comprise one or more additional amino acid sequences that are located on its N-terminus, the C-terminus and/or between the first and the second polypeptide domains or, in case the first polypeptide domain is inserted into the second polypeptide domain or split domains are used, between each part of the respective polypeptide domain fragments.

These additional sequences may each be up to 100 amino acids in length. Besides linker sequences that have no specific functionality, the additional sequences may also be functional peptide sequences, including, without limitation, localization peptide sequences, such as nuclear export signals (NES) or nuclear localization signals (NLS). Such NES or NLS sequences may be derived from viral sequences, such as the HIV NES sequence (LQLPPLERLTL; SEQ ID NO:53) or the SV40 NLS sequence (PKKKRKV; SEQ ID NO:54). The polypeptides of the invention may comprise more than one NES or more than one NLS sequence. The NES or NLS sequence may be located on the N- or C-terminus of the polypeptide. Alternatively, or in addition to the localization signals, the polypeptides may comprise linker sequences to link the first and second polypeptide domain to each other. Suitable linker sequences include the XTEN linker sequence having the amino acid sequence set forth in SEQ ID NO:55, or a GS linker sequence with the sequence set forth in SEQ ID NO:57, or shorter variants of the GS linker that comprise only 2-5 amino acids thereof, such as the peptide GS (Gly-Ser; short GS linker), or combinations of the XTEN and GS linker, such as the sequence set forth in SEQ ID NO:56. It is understood that in case the first polypeptide domain is inserted into the second polypeptide domain, such linkers may be present on both its ends.

In various embodiments, the polypeptides of the invention have a length of up to 1600 amino acids, with the first polypeptide domain being typically up to or equal to 385 amino acids in length, the second polypeptide domain being up to 1090 amino acids in length, such as 967 or 1090 amino acids in length, and the additional sequences present, such as localization signals and linker sequences as defined above, making up the rest, typically about 2 to 200 amino acids in length, preferably 2 to 100 amino acids in length.

In various embodiments, the polypeptides of the invention may have the following structure:
NLS-PPD2.1-L-PPD1-L-PPD2.2-NLS; or
PPD2.2-NLS-L-NLS-PPD2.1-L-PPD1; or
PPD1-L-PPD2-2-NLS-L-NLS-PPD2-1
wherein NLS is a nuclear localization signal, optionally of SEQ ID NO:54; PPD1 is the first polypeptide domain as defined herein; PPD2.1 is the N-terminal part of the second polypeptide domain as defined herein, optionally up to and including residue 338, 655 or 689 using the positional numbering of SEQ ID NO:2, PPD2.2 is the C-terminal part of the second polypeptide domain as defined herein, optionally starting from residue 339, 656 or 690; and L is a linker sequence, optionally selected from SEQ ID NO:55, SEQ ID NO:56 or SEQ ID NO:57 or a combination thereof. In such embodiments, PPD2.1 is preferably the fragment 1-338 and PPD2.2 is the fragment 339-967 (using the numbering of SEQ ID NO:2).

In various embodiments, the polypeptides of the invention may have the following structure:
NLS-PPD2.1-L1-PPD1-L2-PPD2.2-NLS
wherein NLS, PPD1, PPD2.1 and PPD2.2 are as defined above and L1 and L2 are each the amino acid sequence set forth in SEQ ID NO:55, or L1 is the amino acid sequences set forth in SEQ ID NO:57+SEQ ID NO:55 directly linked to each other and L2 is the amino acid sequences set forth in SEQ ID NO:55+SEQ ID NO:57 directly linked to each other.

In various embodiments, the polypeptides of the invention may have the following structure:
PPD2.2-NLS-L1-NLS-PPD2.1-L2-PPD1
wherein NLS, PPD1, PPD2.1 and PPD2.2 are as defined above and L1 is the amino acid sequence set forth in SEQ ID NO:55 and L2 is the amino acid sequence set forth in SEQ ID NO:56.

In various embodiments, the polypeptides of the invention may have the following structure:
PPD1-L-PPD2-2-NLS-L-NLS-PPD2-1
wherein NLS, PPD1, PPD2.1 and PPD2.2 are as defined above and L1 is the amino acid sequence set forth in SEQ ID NO:56 and L2 is the amino acid sequence set forth in SEQ ID NO:55.

In various embodiments of the invention, the isolated polypeptide has the amino acid sequence set forth in any one of SEQ ID NOS: 58-76 or 151-163, for example the sequence set forth in SEQ ID NO:161.

As detailed above, the inventors found that overexpression of ADAR2 deaminase domain will cause off-target editing in the whole transcriptome and that this may be further decreased by splitting the ADAR2 deaminase domain to two parts, and the parts fused to the N-terminal of dCasRx and C-terminal of dCas13b, respectively.

Accordingly, in various embodiments, the present invention relates to a fusion protein comprising a split ADAR2 deaminase domain, as defined herein.

Specifically, in such embodiments, the invention relates to an isolated polypeptide comprising or consisting of
(1) a fragment of a first polypeptide domain, wherein said first polypeptide domain has an amino acid sequence that
   (i) shares at least 70, preferably at least 80, more preferably at least 90%, most preferably at least 95% sequence identity with the amino acid sequence set forth in SEQ ID NO:1 over its entire length; and
   (ii) comprises the amino acid substitution 173Q using the positional numbering of SEQ ID NO:1; and, optionally,
   (iii) comprises amino acid substitutions at any one or more of the positions corresponding to positions 33, 34, 36, 139, 140, 142, 143, 145, 154, 155, 156, 158, 159, 160, 162, and 164 of SEQ ID NO:1;
   wherein
   (a) said fragment is a C-terminal fragment of 230-239 amino acids in length and comprises at least 230 amino acids corresponding to positions 156 to 385 of SEQ ID NO:1; or
   (b) said fragment is an N-terminal fragment of 146-155 amino acids in length and comprises at least 146 amino acids corresponding to positions 1 to 146 of SEQ ID NO:1; and
(2) a second polypeptide domain, wherein the second polypeptide domain comprises an amino acid sequence that shares at least 70, preferably at least 80, more preferably at least 90%, most preferably at least 95% sequence identity with the amino acid sequence set forth in SEQ ID NO:2 over its entire length and comprises the amino acid substitutions 239A, 244A, 858A, and 863A and optionally 940L using the positional numbering of SEQ ID NO:2; and
wherein, if the first polypeptide domain fragment is an N-terminal fragment, it is fused to the C-terminus of the second polypeptide domain, or if the first polypeptide domain fragment is a C-terminal fragment, it is fused to the N-terminus of the second polypeptide domain.

In these embodiments, the fusion protein comprises a fragment of the ADAR2dd (first polypeptide domain) that comprises either at least amino acids 1 to 146 (N-terminal fragment) or at least amino acids 156 to 385 (C-terminal fragment), using the positional numbering of SEQ ID NO:1. The N-terminal fragment may be up to 155 amino acids in length and thus may comprise amino acids 1 to 155 of SEQ ID NO:1. In various embodiments, it comprises amino acids 1 to 147, 148, 149, 150, 151, 152, 153 or 154 using the numbering of SEQ ID NO:1. The C-terminal fragment may be up to 239 amino acids in length and may start from amino acid 147, 148, 149, 150, 151, 152, 153, 154, 155 or 156 and ending with amino acid 385 using the positional numbering of SEQ ID NO:1.

In various embodiments, the fragment of ADAR2dd is a C-terminal fragment. Said C-terminal fragment is preferably the fragment corresponding to amino acids 150-385 using the positional numbering of SEQ ID NO:1. In various embodiments, it consists of the amino acids corresponding to positions 150-385 of SEQ ID NO:1, and may include any one or more of the mutations listed herein for said part of the ADAR2dd, i.e. in particular 173Q and optionally one or more mutations in any amino acid corresponding to positions 154, 155, 156, 158, 159, 160, 162, and 164 of SEQ ID NO:1.

The fusion protein comprising an ADAR2dd fragment further comprises a Cas family polypeptide domain as defined herein (second polypeptide domain). In the afore-mentioned embodiments, this Cas family protein domain is derived from dCasRx having the amino acid sequence set forth in SEQ ID NO:2. All embodiments disclosed above for said second polypeptide domains derived from SEQ ID NO:2 in relation to polypeptides comprising a full ADAR domain, similarly apply to the fusion proteins comprising only part of the ADAR domain. This means that the CasRx domain is inactivated by including the mutations 239A, 244A, 858A, and 863A relative to SEQ ID NO:2. Optionally, they may also include the mutation 940L using the positional numbering of SEQ ID NO:2, for which it was found that it further reduces off-target activity.

In various embodiments, these isolated polypeptides that are fusions of part of the ADAR domain with dCasRx, may comprise or consist of the amino acid sequence set forth in any one of SEQ ID NOS: 77-78.

While the above described fusion proteins are those with a CasRx-derived targeting moiety, the invention also features fusion proteins of ADAR2dd fragments with Cas13b-derived second polypeptide domains. Such isolated polypeptides may comprise or consist of
(1) a fragment of a first polypeptide domain, wherein said first polypeptide sequence has an amino acid sequence that
   (i) shares at least 70, preferably at least 80, more preferably at least 90%, most preferably at least 95% sequence identity with the amino acid sequence set forth in SEQ ID NO:1 over its entire length; and
   (ii) comprises the amino acid substitution 1730 using the positional numbering of SEQ ID NO:1; and, optionally,
   (iii) comprises amino acid substitutions at any one or more of the positions corresponding to positions 33, 34, 36, 139, 140, 142, 143, 145, 154, 155, 156, 158, 159, 160, 162, and 164 of SEQ ID NO:1;
   wherein
   (a) said fragment is a C-terminal fragment of 230-239 amino acids in length and comprises at least 230 amino acids corresponding to positions 156 to 385 of SEQ ID NO:1; or
   (b) said fragment is an N-terminal fragment of 146-155 amino acids in length and comprises at least 146 amino acids corresponding to positions 1 to 146 of SEQ ID NO:1; and
(2) a second polypeptide domain, wherein the second polypeptide domain comprises an amino acid sequence that shares at least 70, preferably at least 80, more preferably at least 90%, most preferably at least 95% sequence identity with the amino acid sequence set forth in SEQ ID NO:3 over its entire length and comprises the amino acid substitutions 133A and 1058A using the positional numbering of SEQ ID NO:3; and
wherein, if the first polypeptide domain fragment is an N-terminal fragment, it is fused to the C-terminus of the second polypeptide domain, or if the first polypeptide domain fragment is a C-terminal fragment, it is fused to the N-terminus of the second polypeptide domain.

In these embodiments, the fusion protein also comprises a fragment of the ADAR2dd (first polypeptide domain) that is defined identical to the ones above, i.e. may comprise either at least amino acids 1 to 146 (N-terminal fragment) or at least amino acids 156 to 385 (C-terminal fragment), using the positional numbering of SEQ ID NO:1. The N-terminal fragment may be up to 155 amino acids in length and thus may comprise amino acids 1 to 155 of SEQ ID NO:1. In various embodiments, it comprises amino acids 1 to 147, 148, 149, 150, 151, 152, 153 or 154 using the numbering of SEQ ID NO:1. The C-terminal fragment may be up to 239 amino acids in length and may start from amino acid 147, 148, 149, 150, 151, 152, 153, 154, 155 or 156 and ending with amino acid 385 using the positional numbering of SEQ ID NO:1.

In various embodiments, if the fusion proteins with CasRx comprise the C-terminal part of ADAR2dd, the fusion proteins with Cas13b comprise the corresponding N-terminal part and vice versa. It is also understood that these fragments may comprise any of the mutations defined herein.

In various embodiments, where the second polypeptide domain is derived from Cas13b (SEQ ID NO:3), the first polypeptide domain is an N-terminal fragment and comprises or consists of the amino acids corresponding to amino acids 1-149 of SEQ ID NO:1. In such embodiments, the first polypeptide domain fragment may comprise an amino acid substitution at the position corresponding to position 145 of SEQ ID NO:1, for example the amino acid substitution 145D, using the positional numbering of SEQ ID NO:1.

These fusion proteins may have a second polypeptide domain based on the amino acid sequence set forth in SEQ ID NO:3, as defined above. All embodiments disclosed above for said second polypeptide domains derived from SEQ ID NO:3 in relation to polypeptides comprising a full ADAR domain, similarly apply to the fusion proteins comprising only part of the ADAR domain. This means that the Cas13b domain is inactivated by including the mutations 133A and 1058A relative to SEQ ID NO:3.

The isolated polypeptides of the invention that are fusion proteins of an ADAR2 domain fragment and dCas13b, as defined herein, may, in various embodiments, have the amino acid sequence set forth in any one of SEQ ID NOS: 79-80.

All isolated polypeptides defined above that comprise a fragment of the ADAR2 domain may, similar to those comprising the full length ADAR2dd, comprise one or more additional amino acid sequences that are located on the N-terminus, the C-terminus and/or between the first and the second polypeptide domains. These additional amino acid sequences may also be selected from nuclear export signals (NES), nuclear localization signals (NLS), and linker sequences, preferably any one of the sequences set forth in SEQ ID NOS: 53-57.

In various embodiments, the fusion protein has the structure (in N- to C-terminal orientation):
PPD1.2-L-NLS-PPD2-NLS
   wherein PPD1.2 is the C-terminal fragment of the first polypeptide domain, L is a linker amino acid sequence, such as the one having the amino acid sequence set forth in SEQ ID NO:55, NLS is a nuclear localization signal, for example the sequence set forth in SEQ ID NO:54, and PPD2 is the second polypeptide domain, as defined herein, preferably a dCasRx domain based on SEQ ID NO:2.

In various embodiments, the fusion protein may have the structure:
NLS-PPD2-NLS-L-PPD1.1 or
PPD2-NES-L-PPD1.1
   wherein PPD1.1 is the N-terminal fragment of the first polypeptide domain, L is a linker amino acid sequence, such as the one having the amino acid sequence set forth in SEQ ID NO:55, NLS is a nuclear localization signal, for example the sequence set forth in SEQ ID NO:54, NES is a nuclear export signal, such as that set forth in SEQ ID NO:53, and PPD2 is the second polypeptide domain, as defined herein, preferably a dCas13b domain based on SEQ ID NO:3.

The polypeptides defined above that comprise a fragment of the ADAR2dd as the first polypeptide domain may be combined with each other such that there are at least two different fusion proteins, one comprising the N-terminal fragment of ADAR2dd and one comprising the C-terminal fragment of ADAR2dd. Preferably, these two fusion proteins are as defined above, with one comprising a dCasRx domain and the other comprising a dCas13b domain. Generally, the fusion proteins are combined such that a fully functional ADAR2dd can be formed by adjacent binding of the two fusion proteins to a target RNA.

The invention thus features compositions comprising at least two polypeptides as defined above, wherein the first polypeptide is the isolated polypeptide comprising a fragment of the first polypeptide domain in combination with a second polypeptide domain based on SEQ ID NO:2 and the second polypeptide is the isolated polypeptide comprising a fragment of the first polypeptide domain that combines with the fragment of the first polypeptide to form the full first polypeptide domain in combination with a second polypeptide domain based on SEQ ID NO:3. In various embodiments, if the first polypeptide comprises the N-terminal fragment of the first polypeptide domain, the second polypeptide comprises the C-terminal fragment of the first polypeptide domain, or wherein if the first polypeptide comprises the C-terminal fragment of the first polypeptide domain, the second polypeptide comprises the N-terminal fragment of the first polypeptide domain.

In addition to the above-described modifications, polypeptides according to the embodiments described herein can comprise amino acid modifications, in particular amino acid substitutions, insertions, or deletions. Such polypeptides are, for example, further developed by targeted genetic modification, i.e. by way of mutagenesis methods, and optimized for specific purposes or with regard to special properties (for example, with regard to their catalytic activity, stability, etc.). If such additional modifications are introduced into the polypeptides of the invention, these preferably do not affect, alter or reverse the mutations detailed above.

In various embodiments, the polypeptides may be post-translationally modified, for example glycosylated. Such modification may be carried out by recombinant means, i.e. directly in the host cell upon production, or may be achieved chemically or enzymatically after synthesis of the polypeptide, for example in vitro.

In various embodiments, the polypeptide may be characterized in that it is obtainable from a polypeptide as described above as an initial molecule by single or multiple conservative amino acid substitution. The term “conservative amino acid substitution” means the exchange (substitution) of one amino acid residue for another amino acid residue, where such exchange does not lead to a change in the polarity or charge at the position of the exchanged amino acid, e.g. the exchange of a nonpolar amino acid residue for another nonpolar amino acid residue. Conservative amino acid substitutions in the context of the invention encompass, for example, G=A=S, I=V=L=M, D=E, N=Q, K=R, Y=F, S=T, G=A=I=V=L=M=Y=F=W=P=S=T. Such changes/modifications are covered by means of the sequence identity/homology levels disclosed above.

In one aspect, the invention also relates to an isolated polypeptide comprising an amino acid sequence that shares at least 70, preferably at least 80, more preferably at least 90%, most preferably at least 95% sequence identity with the amino acid sequence set forth in SEQ ID NO:2 or SEQ ID NO:150 over its entire length (dCasRx) and comprises an amino acid substitution in the position corresponding to position 940 of SEQ ID NO:2, preferably 940L and, optionally any one or more of 239A, 244A, 858A, and 863A, using the positional numbering of SEQ ID NO:2. In various embodiments, said polypeptide comprises the 2, 3 or all 4 of the substitutions 239A, 244A, 858A, and 863A, using the positional numbering of SEQ ID NO:2. The sequence identity to SEQ ID NO:2 or SEQ ID NO:150 may, with the exception of the above-listed substituted positions be at least 80, 81, 82, 83, 84, 85, 86, 87, 88, 89, 90, 91, 92, 93, 94, 95, 96, 97, 98, 99 or 100%. In various embodiments, said isolated polypeptide is not fused to an ADAR deaminase domain, but may be fused to a different polypeptide (domain).

The nucleic acid molecules encoding the polypeptides described herein, as well as a vector containing such a nucleic acid, in particular a copying vector or an expression vector, also form part of the present invention.

These can be DNA molecules or RNA molecules. They can exist as an individual strand, as an individual strand complementary to said individual strand, or as a double strand. With DNA molecules in particular, the sequences of both complementary strands in all three possible reading frames are to be considered in each case. Also, to be considered is the fact that different codons, i.e. base triplets, can code for the same amino acids, so that a specific amino acid sequence can be coded by multiple different nucleic acids. As a result of this degeneracy of the genetic code, all nucleic acid sequences that can encode one of the above-described polypeptides are included in this subject of the invention. The skilled artisan is capable of unequivocally determining these nucleic acid sequences, since despite the degeneracy of the genetic code, defined amino acids are to be associated with individual codons. The skilled artisan can therefore, proceeding from an amino acid sequence, readily ascertain nucleic acids coding for that amino acid sequence. In addition, in the context of nucleic acids according to the present invention one or more codons can be replaced by synonymous codons. This aspect refers in particular to heterologous expression of the polypeptides contemplated herein. For example, every organism, e.g. a host cell of a production strain, possesses a specific codon usage. “Codon usage” is understood as the translation of the genetic code into amino acids by the respective organism. Bottlenecks in protein biosynthesis can occur if the codons located on the nucleic acid are confronted, in the organism, with a comparatively small number of loaded tRNA molecules. Although it codes for the same amino acid, the result is that a codon becomes translated in the organism less efficiently than a synonymous codon that codes for the same amino acid. Because of the presence of a larger number of tRNA molecules for the synonymous codon, the latter can be translated more efficiently in the organism.

By way of methods commonly known today such as, for example, chemical synthesis or the polymerase chain reaction (PCR) in combination with standard methods of molecular biology or protein chemistry, a skilled artisan has the ability to manufacture, on the basis of known DNA sequences and/or amino acid sequences, the corresponding nucleic acids all the way to complete genes. Such methods are known, for example, from Sambrook, J., Fritsch, E. F., and Maniatis, T, 2001, Molecular cloning: a laboratory manual, 3rd edition, Cold Spring Laboratory Press.

“Vectors” are understood for purposes herein as elements—made up of nucleic acids—that contain a nucleic acid contemplated herein as a characterizing nucleic acid region. They enable said nucleic acid to be established as a stable genetic element in a species or a cell line over multiple generations or cell divisions. In particular when used in bacteria, vectors are special plasmids, i.e. circular genetic elements. In the context herein, a nucleic acid as contemplated herein is cloned into a vector. Included among the vectors are, for example, those whose origins are bacterial plasmids, viruses, or bacteriophages, or predominantly synthetic vectors or plasmids having elements of widely differing derivations. Using the further genetic elements present in each case, vectors are capable of establishing themselves as stable units in the relevant host cells over multiple generations. They can be present extrachromosomally as separate units, or can be integrated into a chromosome respectively into chromosomal DNA.

Expression vectors encompass nucleic acid sequences which are capable of replicating in the host cells, by preference microorganisms, particularly preferably bacteria, that contain them, and expressing therein a contained nucleic acid. In various embodiments, the vectors described herein thus also contain regulatory elements that control expression of the nucleic acids encoding a polypeptide of the invention. Expression is influenced in particular by the promoter or promoters that regulate transcription. Expression can occur in principle by means of the natural promoter originally located in front of the nucleic acid to be expressed, but also by means of a host-cell promoter furnished on the expression vector or also by means of a modified, or entirely different, promoter of another organism or of another host cell. In the present case at least one promoter for expression of a nucleic acid as contemplated herein is made available and used for expression thereof. Expression vectors can furthermore be regulated, for example by way of a change in culture conditions or when the host cells containing them reach a specific cell density, or by the addition of specific substances, in particular activators of gene expression. One example of such a substance is the galactose derivative isopropyl-beta-D-thiogalactopyranoside (IPTG), which is used as an activator of the bacterial lactose operon (lac operon). In contrast to expression vectors, the contained nucleic acid is not expressed in cloning vectors.

In a further aspect, the invention is also directed to a host cell, preferably a non-human host cell, containing a nucleic acid as contemplated herein or a vector as contemplated herein. A nucleic acid as contemplated herein or a vector containing said nucleic acid is preferably transformed into a microorganism, which then represents a host cell according to an embodiment. Methods for the transformation of cells are established in the existing art and are sufficiently known to the skilled artisan. All cells are in principle suitable as host cells, i.e. prokaryotic or eukaryotic cells. Those host cells that can be manipulated in genetically advantageous fashion, e.g. as regards transformation using the nucleic acid or vector and stable establishment thereof, are preferred, for example single-celled fungi or bacteria. In addition, preferred host cells are notable for being readily manipulated in microbiological and biotechnological terms. This refers, for example, to easy culturability, high growth rates, low demands in terms of fermentation media, and good production and secretion rates for foreign proteins. The polypeptides can furthermore be modified, after their manufacture, by the cells producing them, for example by the addition of sugar molecules, formylation, amination, etc. Post-translation modifications of this kind can functionally influence the polypeptide

Further embodiments are represented by those host cells whose activity can be regulated on the basis of genetic regulation elements that are made available, for example, on the vector, but can also be present a priori in those cells. They can be stimulated to expression, for example, by controlled addition of chemical compounds that serve as activators, by modifying the culture conditions, or when a specific cell density is reached. This makes possible economical production of the proteins contemplated herein. One example of such a compound is IPTG, as described earlier.

Host cells can be prokaryotic or bacterial cells, such as E. coli cells. Bacteria are notable for short generation times and few demands in terms of culturing conditions. As a result, economical culturing methods respectively manufacturing methods can be established. In addition, the skilled artisan has ample experience in the context of bacteria in fermentation technology. Gram-negative or Gram-positive bacteria may be suitable for a specific production instance, for a wide variety of reasons to be ascertained experimentally in the individual case, such as nutrient sources, product formation rate, time requirement, etc. In various embodiments, the host cells may be E. coli cells.

Host cells contemplated herein can be modified in terms of their requirements for culture conditions, can comprise other or additional selection markers, or can also express other or additional proteins. They can, in particular, be those host cells that transgenically express multiple proteins or enzymes.

The host cell can, however, also be a eukaryotic cell, which is characterized in that it possesses a cell nucleus. A further embodiment is therefore represented by a host cell which is characterized in that it possesses a cell nucleus. In contrast to prokaryotic cells, eukaryotic cells are capable of post-translationally modifying the protein that is formed. Examples thereof are fungi such as Actinomycetes, or yeasts such as Saccharomyces or Kluyveromyces or insect cells, such as Sf9 cells. This may be particularly advantageous, for example, when the proteins, in connection with their synthesis, are intended to experience specific modifications made possible by such systems. Among the modifications that eukaryotic systems carry out in particular in conjunction with protein synthesis are, for example, the bonding of low-molecular-weight compounds such as membrane anchors or oligosaccharides. In various embodiments, the host cells are thus eukaryotic cells, such as insect cells, for example Sf9 cells.

The host cells contemplated herein are cultured and fermented in a usual manner, for example in discontinuous or continuous systems. In the former case a suitable nutrient medium is inoculated with the host cells, and the product is harvested from the medium after a period of time to be ascertained experimentally. Continuous fermentations are notable for the achievement of a flow equilibrium in which, over a comparatively long period of time, cells die off in part but are also in part renewed, and the protein formed can simultaneously be removed from the medium.

Host cells contemplated herein are preferably used to manufacture the polypeptides described herein.

A further aspect of the invention is therefore a method for manufacturing a polypeptide as described herein, comprising culturing a host cell contemplated herein; and isolating the polypeptide from the culture medium or from the host cell. Culture conditions and mediums can be selected by those skilled in the art based on the host organism used by resorting to general knowledge and techniques known in the art.

The isolated polypeptides described herein, including those comprising the full length ADAR2dd and those comprising only a fragment thereof, may be combined with at least one guide RNA (gRNA) molecule. The gRNA molecule facilitates target RNA recognition, binding and editing in that it—together with the Cas family protein domain—directs the fusion protein to its target RNA site. The invention is thus also directed to a composition comprising any one or more of the polypeptides of the invention, including the compositions/combinations of two polypeptides each comprising part of the ADAR2dd, and at least one gRNA molecule.

In various embodiments, the gRNA molecule comprises a sequence that forms a stem-loop structure and a spacer sequence directly linked to one end of the stem forming sequence. More specifically, the gRNA molecule comprises
(1) a target-specific antisense sequence (spacer sequence) that is at least 24 nucleotides in length and comprises a mismatch C nucleotide at the position that base-pairs with the A to be edited in the target sequence; and
(2) a Cas-binding sequence that is at least 26 nucleotides in length and is recognized and bound by the second polypeptide domain, wherein said sequence has a level of self-complementarity such that a stem-loop structure is formed.

“Base-pairs”, as used in this context, refers to Watson-Crick base-pairing of RNA molecules, i.e. G-C and A-U. The target-specific sequence comprises an RNA antisense sequence that hybridizes to the target sequence by such Watson-Crick base-pairing and may have high complementarity or even full complementarity with the exception of the target A in the target sequence which is mismatched with C to facilitate the deaminase activity of the ADAR2dd. In order to avoid off-target editing of additional A nucleobases in the target sequence, the gRNA molecule may in the target-specific sequence comprise additional mismatches where said additional A nucleotides in the target sequence are mismatched with G in the gRNA. Accordingly, in various embodiments, the target-specific sequence comprises one or more mismatch G nucleotides at sites that (base-)pair with A nucleotides in the target sequence. These off-targets are also referred to as “cis off-targets” and are typically located closer to the nearest terminus relative to the mismatch site. In various embodiments, the number of said additional G-A mismatches in the spacer sequence is 1, 2 or more, preferably 1 or 2.

Generally, the target-specific sequence has little to no self-complementarity to avoid formation of secondary structures that could interfere with target recognition and binding. Said part of the gRNA molecule is thus single-stranded.

The target-specific sequence may be located 3′ to the Cas-binding sequence. This means that it is connected to the 3′ end of the sequence forming the stem-loop structure. Alternatively, it may be located 5′ to the Cas-binding sequence, i.e. it is connected to the 5′ end of the sequence forming the stem-loop structure.

In various embodiments, the mismatch site in the target-specific antisense sequence is located at least 6 nucleotides away from the nearest terminus of the gRNA, for example 7 or more nucleotides. This distance is also referred to as “mismatch distance”. The mismatch site may be located 6 or more nucleotides down- or upstream of the connection point to the double-stranded Cas-binding part, i.e. the stem. Typical distances may be 11 nucleotides, 22 nucleotides, 40 nucleotides, depending on the length of the spacer sequence. For spacer sequences in the range of 20 to 30 nucleotides, for example 26 or 27 nucleotides, the mismatch sequence may, for example, be 7 to 15 nucleotides, such as 8-14 nucleotides or 9-13 nucleotides, or 10-12 nucleotides or 11 nucleotides. For longer spacers, such as spacers of more than 30 and up to 50 or 55 nucleotides in length, the mismatch distance may be greater, for example 11 to 40 nucleotides, such as 22 to 30 nucleotides, for example 23-28 nucleotides, for example 25 nucleotides. A mismatch distance of more than 30 and up to 40 nucleotides is however preferably used in gRNA dimers, as disclosed below.

The total length of the gRNA molecule may be up to 150 nucleotides, preferably up to 100 nucleotides, even more preferably up to 90 nucleotides, or up to 81 nucleotides. The total length refers to the sum of the length of the Cas-binding sequence, i.e. the stem-loop structure, and the length of the target-specific sequence, also referred to as “spacer” sequence. The stem loop-structure is typically about 26 nucleotides in length, for example 30 or 32 to 40 nucleotides, and the spacer length may vary from 24 to about 55 nucleotides. The minimum total length of the gRNA is typically about 50 nucleotides. Typical lengths of the spacer sequence are 25 to 30 nucleotides. However, the inventors have found that under certain circumstances extended spacer sequences having more than 30 nucleotides, for example up to 50 nucleotides may be advantageous. The length of the spacer sequence also correlates with the desired mismatch distance, as the mismatch is preferably at least 6 nucleotides away from the nearest terminus.

The Cas-binding sequence is typically about 30 nucleotides in length, for example about 26 to 40 nucleotides, such as 36 nucleotides. The stem-structure may be about 8 to 16 nucleotides in length, for example about 14 nucleotides, while the loop structure may be 2 to 10 nucleotides in length, for example 8 nucleotides. The two sequence parts forming the stem have enough complementarity to hybridize to each other under conditions of use, i.e. typically under conditions as encountered in a cell, including the cytoplasm and the nucleus. These two sequence parts forming the stem flank the unpaired sequence forming the loop. The spacer sequence is typically directly connected to one of the stem-forming sequences. Optionally, the other stem-forming sequence not connected to the spacer may also be extended by a sequence that does not form an intermolecular double-stranded structure. Said sequence may be another spacer sequence that has target complementarity and extends, relatively to the first spacer sequence, in the other direction of the target molecule. However, said second spacer sequence does typically not contain a C-A mismatch, wherein the position in the spacer sequence pairing with an A in the target sequence is occupied by a C. The second spacer may however contain G-A mismatches, where the positions pairing with A in the target sequence are occupied by G to avoid off-target editing.

Accordingly, the gRNA may comprise two target-specific sequences that flank the Cas-binding sequence (2), wherein preferably one of the two target-specific sequences is free of mismatches, i.e. of C-A mismatches, and the other is the target-specific sequence (1).

In various embodiments, the gRNA may be a dimer in that it comprises two gRNA units linked to each other, for example by a phosphodiester bond. In various embodiments, the two units differ in that one unit is a gRNA molecule as defined above and the other is linked to it upstream (to its 3′ end) or downstream (to its 5′ end) but contains no mismatch for ADAR2dd-mediated editing. The two units are preferably designed such that they hybridize to adjacent parts in the target sequence and thus recruit two polypeptides of the invention (Cas-ADAR2 fusion proteins). Specifically, the gRNA molecule as defined above may be linked to a second gRNA molecule that comprises
(1) a target-specific antisense sequence that is at least 24 nucleotides in length; and
(2) a Cas-binding sequence that is at least 30 nucleotides in length and is recognized and bound by the second polypeptide domain, wherein said sequence has a level of self-complementarity such that a stem-loop structure is formed.

The two units of the gRNA dimer may be part of a single nucleotide sequence and thus are typically linked by a phosphodiester bond. As noted above, the two gRNA molecules (unity) may differ in that one of the two molecules does not comprise a C mismatch in the target-complementary sequence. In addition, and in various embodiments, they may also differ in their Cas-binding sequences.

In such dimers, the orientation of the two units may be such that the mismatch site is between the two Cas-binding sequences. It may be arranged closer to one of those two stem-loop-structures, such as having a mismatch distance of 11, or may be located in the middle between the two, for example having a mismatch distance of 40. The location in the middle between the two Cas-binding sequences has the advantage that it becomes accessible for both ADAR2dd units of the fusion proteins binding to the two Cas-binding sequences. This can significantly increase the editing level relative to a “normal” monomeric gRNA.

In various embodiments, the gRNA comprising two units comprises two Cas-binding sequences that recruit dCasRx. These may be identical. This allows to recruit two fusion proteins of the invention and thus increase editing efficiency, as two ADAR2 deaminase domains are brought in close proximity of the target site.

As dCasRx can process its own gRNA, which will cleave and separate the extended gRNA (dimeric gRNA), the CasRx domain preferably contains the 940L mutation that was shown to abolish pre-CrRNA cleavage (Konermann et al., supra).

In various embodiments, the gRNA comprising two units comprises two Cas-binding sequences, one for recruiting dCasRx and the other for recruiting dCas13b. It was found that the recruitment of dCas13b in addition to a dCasRx-based fusion protein of the invention may help in editing some target cites in reporter assays and endogenous genes. Furthermore, said gRNA also allowed efficient editing in the cytoplasm due to the improvement of the compatibility with a NES, as facilitated by the help of dCas13b.

The compositions of the invention that comprise one or more polypeptides of the invention in combination with at least one gRNA, with the gRNA being functional with the polypeptides comprised in the composition, may be used for targeted editing of RNA in a cell, either in vitro or in vivo.

The targeted RNA that is edited may, in various embodiments, be mRNA. Suitable mRNAs that may be targeted by the compositions of the invention include, without limitation,
(1) the mRNA coding for the cell surface receptor angiotensin-converting enzyme 2 (ACE2);
(2) the mRNA coding for the cellular protease TMPRSS2 (transmembrane protease serine 2 isoform 2);
(3) the mRNA coding for the voltage-gated sodium channel Nav1.4 (SCN4A); and
(4) the mRNA transcript of the keratin 5 (KRT5) or keratin 14 (KRT14) gene.

The sequences of these target genes may be those set forth in SEQ ID NO:81 (ACE2), SEQ ID NO:82 (TMPRSS2); SEQ ID NO:83 (KRT14) and SEQ ID NO:84 (SCN4A).

In these embodiments, the gRNA may target the codons coding for K31 or K353 of ACE2 receptor, the codon coding for S441 of TMPRSS2, the codon coding for K1244 of SCN4A, or the codon coding for R125 of keratin. It is to be understood that these target transcripts and the specified sites are proof-of-concept targets, but that the compositions and methods of the present invention can be adapted to edit numerous other targets and sites.

Various gRNA sequences that have been used in accordance with the invention are those that are obtained by transcription of the DNA sequences set forth in SEQ ID NOS: 85-132.

The invention also features compositions that comprise at least one nucleic acid sequence or molecule encoding at least one polypeptide of the invention, optionally in combination with a gRNA or a nucleic acid sequence or molecule coding for said gRNA. The nucleic acid sequence coding for the polypeptide of the invention and the nucleic acid coding for the gRNA may be on the same or separate molecules.

The invention is also directed to pharmaceutical compositions comprising the isolated polypeptides of the invention or the nucleic acid encoding them or the compositions of the invention and further comprising one or more of diluents, stabilizers, excipients and carriers.

The isolated polypeptides of the invention, the nucleic acids encoding them or the compositions of the invention may be for use as a pharmaceutical. The invention is thus also directed to the use of the isolated polypeptides of the invention, the nucleic acids encoding them or the compositions of the invention for targeted RNA editing. Said targeted RNA editing may be in vitro, for example in cultured cells, or may be in vivo. Examples of targeted RNAs have been disclosed above, but the invention is not limited thereto.

The invention is also directed to methods for targeted editing of the RNA in a cell, comprising introducing into said cell the isolated polypeptide of the invention, a nucleic acid encoding it, or the composition of the invention. Such methods may be for the treatment or prevention of a disease or disorder caused by RNA, for example an aberrant RNA transcript or pathogenic RNA, such as viral RNA.

These methods may for example be used for the treatment or prevention of SARS-CoV-2 infection, comprising administering a therapeutically or prophylactically effective amount of a composition of the invention that targets the mRNA coding for the cell surface receptor angiotensin-converting enzyme 2 (ACE2) or the cellular protease TMPRSS2 (transmembrane protease serine 2 isoform 2), in particular the codons coding for K31 or K353 of ACE2 receptor or the codon coding for S441 of TMPRSS2, to a subject in need thereof.

In alternative embodiments, the methods may be used for the treatment or prevention of pain (pain management), comprising administering a therapeutically or prophylactically effective amount of a composition of the invention that targets the mRNA coding for the voltage-gated sodium channel Nav1.4 (SCN4A), in particular the codon coding for K1244 of SCN4A, to a subject in need thereof.

In still alternative embodiments, the methods may be used for the treatment or prevention of epidermolysis bullosa, comprising administering a therapeutically or prophylactically effective amount of a composition of the invention that targets the mRNA coding for keratin 5 or keratin 14, in particular the codon coding for R125 of keratin, to a subject in need thereof.

In all the above methods, the subject may be a human.

All embodiments disclosed herein in relation to the polypeptides and nucleic acids are similarly applicable to the compositions, uses and methods described herein and vice versa.

The invention is further illustrated by the following non-limiting examples and the appended claims.

### EXAMPLES

Materials and Methods

### Example 1

Design and Cloning of Constructs

The gRNA expression plasmids were generated using as backbones pC0043 (Addgene #103864) for Cas13b and pXR003 (Addgene #109053) for CasRx. First, pC0043 or pXR003 was digested with Bbsl-HF (New England Biolabs) and gel extracted. Second, reverse complementary single-stranded DNA oligonucleotides containing the relevant spacer sequences were ordered from Integrated DNA Technologies (IDT). Third, the oligonucleotides were phosphorylated, annealed together, and then ligated into the digested plasmids using T4 DNA Ligase (New England Biolabs).

To generate the various mutant constructs in our study, site-directed mutagenesis using the QuikChange Lightning Site-Directed Mutagenesis Kit (Agilent #210519) was carried out. The primers for all the missense mutations were designed using the online QuikChange Primer Design program (https://www.agilent.com/store/primerDesignProgram.jsp). Mutagenesis was performed on a sub-cloned human ADAR2(E488Q) deaminase vector. For the luciferase reporters, the W60X (SEQ ID NO:164), W104X (SEQ ID NO:165), W153X (SEQ ID NO:166), or W219X (SEQ ID NO:167) mutation was directly introduced into the Renilla luciferase gene in the psi-check2 plasmid. All cloned constructs were sequence-verified before use.

Cell Culture

Cell culture experiments were performed using HEK293FT, HeLa, and HCT116 human cell lines, which were cultured using Dulbecco's Modified Eagle Medium (DMEM) with high glucose (Hyclone), supplemented with 10% fetal bovine serum (FBS) (Hyclone), 1× L-glutamine (Gibco), and 0.2× penicillin-streptomycin (Gibco). To introduce constructs into the cells, 1.8×10⁵ cells were seeded in a 24-well plate one day prior to transfection to reach ˜70% confluency the next day. 300 ng of gRNA plasmid was co-transfected with 300 ng of dCas13b-ADAR2 or dCasRx-ADAR2 plasmid using jetPRIME transfection reagent according to manufacturer's instructions. RNA was harvested 48h post transfection. For luciferase assays, 3.6×10⁴ cells were seeded in 96-well white plate one day prior to transfection. Subsequently, 58 ng of gRNA plasmid was co-transfected with 58 ng of dCas13b-ADAR2 or dCasRx-ADAR2 plasmid and 4 ng of luciferase reporter plasmid using jetPRIME transfection reagent.

RNA Isolation and cDNA Synthesis

RNA was either lysed using TRizol (Invitrogen), then further isolated using Direct-zol RNA Miniprep kit (Zymo Research), or by using RNAzol (Molecular Research Center) according to manufacturer's instructions. 500 ng to 1 ug of RNA was used for cDNA synthesis using qScript cDNA Supermix (Quantabio). RNA samples were treated with DNasel (New England Biolabs) before cDNA synthesis when using RNAzol as the extraction method.

Assessment of RNA Editing in Human Cells

The extent of programmable RNA editing was assessed using three different methods:
(1) Luciferase assay: The luciferase activity was measured 48h post transfection using the Promaga dual luciferase assay kit according to manufacturer's instructions in a Promega Glomax Multi Detection Plate Reader.
(2) Sanger sequencing: The target loci were amplified by PCR using reverse transcribed cDNA and 05 High-Fidelity DNA Polymerase (New England Biolabs). The PCR products were extracted from a 2% agarose gel using PureNA Gel Extraction kit (Research Instruments) and then sent for Sanger sequencing by Axil Scientific.
(3) Next generation sequencing: Sequencing libraries were constructed via two rounds of PCR. In the first round, the loci-of-interest were amplified from reverse transcribed cDNA using 05 High-Fidelity DNA Polymerase (New England Biolabs). Each forward primer contains the common sequence GCG TTA TCG AGG TCN NNN (SEQ ID NO:168), while each reverse primer contains the common sequence GTG CTC TTC CGA TCT (SEQ ID NO:169). In the second round, the PCR products from the first round were barcoded with the following primers: forward, AAT GAT ACG GCG ACC ACC GAG ATC TAC ACC CTA CAC GAG CGT TAT CGA GGT C (SEQ ID NO:170); reverse, CAA GCA GAA GAC GGC ATA CGA GAT (barcode) GTG ACT GGA GTT CAG ACG TGT GCT CTT CCG ATC T (SEQ ID NO:171). 10-bp barcodes designed by Fluidigm for the Access Array System were used. All samples were sequenced on NextSeq or HiSeq (Illumina) to produce paired 151-bp reads.

1. Isolated polypeptide comprising or consisting of
(1) a first polypeptide domain comprising an amino acid sequence that
(i) shares at least 70, preferably at least 80, more preferably at least 90%, most preferably at least 95% sequence identity with the amino acid sequence set forth in SEQ ID NO:1 over its entire length; and
(ii) comprises the amino acid substitution 173Q using the positional numbering of SEQ ID NO:1; and, optionally,
(iii) comprises amino acid substitutions at any one or more of the positions corresponding to positions 145, 33, 34, 36, 139, 140, 142, 143, 154, 155, 156, 158, 159, 160, 162, and 164 of SEQ ID NO:1 (hADAR2dd);
(2) a second polypeptide domain comprising an amino acid sequence that shares at least 70, preferably at least 80, more preferably at least 90%, most preferably at least 95% sequence identity with
(i) the amino acid sequence set forth in SEQ ID NO:2 over its entire length (dCasRx) and comprises the amino acid substitutions 239A, 244A, 858A, and 863A using the positional numbering of SEQ ID NO-2; or
(ii) the amino acid sequence set forth in SEQ ID NO:3 over its entire length (dCas13b) and comprises the amino acid substitutions 133A and 1058A, using the positional numbering of SEQ ID NO:3;
wherein the first polypeptide domain is fused to the second polypeptide domain or inserted into the second polypeptide domain;
with the proviso that if the second polypeptide domain comprises an amino acid sequence that shares at least 70, preferably at least 80, more preferably at least 90%, most preferably at least 95% sequence identity with the amino acid sequence set forth in SEQ ID NO:3 and comprises the amino acid substitutions 133A and 1058A, using the positional numbering of SEQ ID NO:3, the first polypeptide domain does not have the amino acid sequence of SEQ ID NO:1 with the amino acid substitution 173Q in combination with one of 33E, 36L, 140G/S/E, 158D, 159E, 1600, and 162E.

2. The isolated polypeptide of claim 1, wherein the first polypeptide domain comprises an amino acid substitution at the position corresponding to position 145 of SEQ ID NO:1.

3. The isolated polypeptide of claim 1 or 2, wherein the first polypeptide domain comprises any one or more of the amino acid substitutions 33G, 33A, 33E, 34G, 36L, 139C, 140A, 140D, 142Y, 143A, 145A, 145D, 154A, 155A, 155D, 156A, 158G, 158L, 159A, 159D, 160A, 160D, 160E, 160L, 162A, 164L, and 164V, using the positional numbering of SEQ ID NO:1.

4. The isolated polypeptide of any one of claims 1 to 3, wherein the first polypeptide domain comprises the amino acid substitution 145D, using the positional numbering of SEQ ID NO:1.

5. The isolated polypeptide of any one of claims 1 to 4, wherein the first polypeptide domain has the amino acid sequence set forth in any one of SEQ ID NOS:4-49.

6. The isolated polypeptide of any one of claims 1 to 5, wherein the second polypeptide domain comprises an amino acid substitution in the position corresponding to position 940 of SEQ ID NO:2, preferably 940L.

7. The isolated polypeptide of any one of claims 1 to 6, wherein the second polypeptide domain has the amino acid sequence set forth in any one of SEQ ID NOS:50-52.

8. The isolated polypeptide of any one of claims 1 to 7, wherein the first polypeptide domain is located C-terminally to the second polypeptide domain.

9. The isolated polypeptide of claim 8, wherein the first polypeptide domain is fused to the C-terminus of the second polypeptide domain.

10. The isolated polypeptide of any one of claims 1 to 7, wherein the first polypeptide domain is inserted into the second polypeptide domain.

11. The isolated polypeptide of claim 10, wherein the first polypeptide domain is inserted after position 338, 655 or 689 of the second polypeptide domain, using the positional numbering of SEQ ID NO:2.

12. The isolated polypeptide of claim 11, wherein the first polypeptide domain is inserted after position 338 of the second polypeptide domain, using the positional numbering of SEQ ID NO:2.

13. The isolated polypeptide of any one of claims 1 to 12, wherein the isolated polypeptide comprises one or more additional amino acid sequences that are located on the N-terminus, the C-terminus and/or between the first and the second polypeptide domains.

14. The isolated polypeptide of claim 13, wherein the one or more additional amino acid sequences are selected from nuclear export signals (NES), nuclear localization signals (NLS), and linker sequences, preferably any one of the sequences set forth in SEQ ID NOS: 53-57.

15. The isolated polypeptide of any one of claims 1 to 14, wherein the polypeptide has the amino acid sequence set forth in any one of SEQ ID NOS: 58-76.

16. An isolated polypeptide comprising or consisting of
(1) a fragment of a first polypeptide domain, wherein said first polypeptide domain has an amino acid sequence that
(i) shares at least 70, preferably at least 80, more preferably at least 90%, most preferably at least 95% sequence identity with the amino acid sequence set forth in SEQ ID NO:1 over its entire length; and
(ii) comprises the amino acid substitution 1730 using the positional numbering of SEQ ID NO:1; and, optionally,
(iii) comprises amino acid substitutions at any one or more of the positions corresponding to positions 33, 34, 36, 139, 140, 142, 143, 145, 154, 155, 156, 158, 159, 160, 162, and 164 of SEQ ID NO:1;
wherein
(c) said fragment is a C-terminal fragment of 230-239 amino acids in length and comprises at least 230 amino acids corresponding to positions 156 to 385 of SEQ ID NO:1; or
(d) said fragment is an N-terminal fragment of 146-155 amino acids in length and comprises at least 146 amino acids corresponding to positions 1 to 146 of SEQ ID NO:1; and
(2) a second polypeptide domain, wherein the second polypeptide domain comprises an amino acid sequence that shares at least 70, preferably at least 80, more preferably at least 90%, most preferably at least 95% sequence identity with the amino acid sequence set forth in SEQ ID NO:2 over its entire length and comprises the amino acid substitutions 239A, 244A, 858A, and 863A and optionally 940L using the positional numbering of SEQ ID NO-2; and
wherein, if the first polypeptide domain fragment is an N-terminal fragment, it is fused to the C-terminus of the second polypeptide domain, or if the first polypeptide domain fragment is a C-terminal fragment, it is fused to the N-terminus of the second polypeptide domain.

17. The isolated polypeptide of claim 16, wherein the first polypeptide domain fragment is a C-terminal fragment and comprises or consists of the amino acids corresponding to amino acids 150-385 of SEQ ID NO:1.

18. The isolated polypeptide of claim 16 or 17, wherein the polypeptide has the amino acid sequence set forth in any one of SEQ ID NOS: 77-78.

19. An isolated polypeptide comprising or consisting of
(1) a fragment of a first polypeptide domain, wherein said first polypeptide domain has an amino acid sequence that
(i) shares at least 70, preferably at least 80, more preferably at least 90%, most preferably at least 95% sequence identity with the amino acid sequence set forth in SEQ ID NO:1 over its entire length; and
(ii) comprises the amino acid substitution 1730 using the positional numbering of SEQ ID NO:1; and, optionally,
(iii) comprises amino acid substitutions at any one or more of the positions corresponding to positions 33, 34, 36, 139, 140, 142, 143, 145, 154, 155, 156, 158, 159, 160, 162, and 164 of SEQ ID NO:1;
wherein
(a) said fragment is a C-terminal fragment of 230-239 amino acids in length and comprises at least 230 amino acids corresponding to positions 156 to 385 of SEQ ID NO:1; or
(b) said fragment is an N-terminal fragment of 146-155 amino acids in length and comprises at least 146 amino acids corresponding to positions 1 to 146 of SEQ ID NO:1; and
(2) a second polypeptide domain, wherein the second polypeptide domain comprises an amino acid sequence that shares at least 70, preferably at least 80, more preferably at least 90%, most preferably at least 95% sequence identity with the amino acid sequence set forth in SEQ ID NO:3 over its entire length and comprises the amino acid substitutions 133A and 1058A using the positional numbering of SEQ ID NO:3; and
wherein, if the first polypeptide domain fragment is an N-terminal fragment, it is fused to the C-terminus of the second polypeptide domain, or if the first polypeptide domain fragment is a C-terminal fragment, it is fused to the N-terminus of the second polypeptide domain.

20. The isolated polypeptide of claim 19, wherein the first polypeptide domain comprises an amino acid substitution at the position corresponding to position 145 of SEQ ID NO:1.

21. The isolated polypeptide of claim 19 or 20, wherein the first polypeptide domain is an N-terminal fragment and comprises or consists of the amino acids corresponding to amino acids 1-149 of SEQ ID NO:1

22. The isolated polypeptide of any one of claims 19 to 21, wherein the first polypeptide domain comprises the amino acid substitution 145D, using the positional numbering of SEQ ID NO:1.

23. The isolated polypeptide of any one of claims 19 to 22, wherein the polypeptide has the amino acid sequence set forth in any one of SEQ ID NOS: 79-80.

24. The isolated polypeptide of any one of claims 16 to 23, wherein the isolated polypeptide comprises one or more additional amino acid sequences that are located on the N-terminus, the C-terminus and/or between the first and the second polypeptide domains.

25. The isolated polypeptide of claim 24, wherein the one or more additional amino acid sequences are selected from nuclear export signals (NES), nuclear localization signals (NLS), and linker sequences, preferably any one of the sequences set forth in SEQ ID NOS: 53-57.

26. Composition comprising at least two polypeptides, wherein the first polypeptide is the isolated polypeptide of any one of claims 16 to 18, 24 and 25 and the second polypeptide is the isolated polypeptide of any one of claims 19 to 25, wherein if the first polypeptide comprises the N-terminal fragment of the first polypeptide domain, the second polypeptide comprises the C-terminal fragment of the first polypeptide domain, or wherein if the first polypeptide comprises the C-terminal fragment of the first polypeptide domain, the second polypeptide comprises the N-terminal fragment of the first polypeptide domain.

27. Composition comprising the isolated polypeptide of any one of claims 1 to 15 or the composition of claim 26 and further comprising a guide RNA (gRNA) molecule.

28. The composition of claim 27, wherein the gRNA molecule comprises
(1) a target-specific antisense sequence (spacer sequence) that is at least 24 nucleotides in length and comprises a mismatch C nucleotide at the position that base-pairs with the A to be edited in the target sequence; and
(2) a Cas-binding sequence that is at least 26 nucleotides in length and is recognized and bound by the second polypeptide domain, wherein said sequence has a level of self-complementarity such that a stem-loop structure is formed.

29. The composition of claim 28, wherein the target-specific sequence is located 3′ relative to the Cas-binding sequence.

30. The composition of claim 28, wherein the target-specific sequence is located 5′ relative to the Cas-binding sequence.

31. The composition of any one of claims 28 to 30, wherein the mismatch site is located at least 6 nucleotides away from the nearest terminus of the gRNA.

32. The composition of any one of claims 28 to 31, wherein the target-specific sequence comprises one or more mismatch G nucleotides at sites that pair with A nucleotides in the target sequence.

33. The composition of any one of claims 28 to 32, wherein the gRNA comprises two target-specific sequences that flank the Cas-binding sequence (2), wherein preferably one of the two target-specific sequences is free of mismatches and the other is the target-specific sequence (1).

34. The composition of any one of claims 28 to 33, wherein the gRNA molecule is up to 100 nucleotides in length.

35. The composition of any one of claims 28 to 34, wherein the gRNA molecule is linked to a second gRNA molecule that comprises
(1) a target-specific antisense sequence that is at least 24 nucleotides in length; and
(2) a Cas-binding sequence that is at least 26 nucleotides in length and is recognized and bound by the second polypeptide domain, wherein said sequence has a level of self-complementarity such that a stem-loop structure is formed.

36. The composition of claim 35, wherein the two gRNA molecules are linked by a phosphodiester bond.

37. The composition of claim 35 or 36, wherein the two gRNA molecules differ in that one of the two molecules does not comprise a C mismatch in the target-complementary sequence.

38. The composition of any one of claims 35 to 37, wherein the two gRNA molecules differ in the Cas-binding sequence.

39. The composition of any one of claims 27 to 38, wherein the gRNA targets the mRNA coding for the cell surface receptor angiotensin-converting enzyme 2 (ACE2).

40. The composition of claim 39, wherein the gRNA targets the codons coding for K31 or K353 of ACE2 receptor.

41. The composition of any one of claims 27 to 38, wherein the gRNA targets the mRNA coding for the cellular protease TMPRSS2.

42. The composition of claim 41, wherein the gRNA targets the codon coding for S441 of TMPRSS2.

43. The composition of any one of claims 27 to 38, wherein the gRNA targets the mRNA coding for the voltage-gated sodium channel Nav1.4 (SCN4A).

44. The composition of claim 43, wherein the gRNA targets the codon coding for K1244 of SCN4A.

45. The composition of any one of claims 27 to 38, wherein the gRNA targets the mRNA transcript of the keratin 5 (KRT5) or keratin 14 (KRT14) gene.

46. The composition of claim 45, wherein the gRNA targets the codon coding for R125 of keratin.

47. Pharmaceutical composition comprising the isolated polypeptide of any one of claims 1 to 25 or the composition of any one of claims 26 to 46 and one or more of diluents, stabilizers, excipients and carriers.

48. The isolated polypeptide of any one of claims 1 to 25 or the composition of any one of claims 26 to 46 for use as a pharmaceutical.

49. Use of the isolated polypeptide of any one of claims 1 to 25 or the composition of any one of claims 26 to 46 for targeted RNA editing.

50. Method for targeted editing of the RNA of a cell, comprising introducing into said cell the isolated polypeptide of any one of claims 1 to 25 or the composition of any one of claims 26 to 46.

51. Method for the treatment or prevention of SARS-CoV-2 infection, comprising administering a therapeutically or prophylactically effective amount of a composition of any one of claims 39-42 to a subject in need thereof.

52. Method for the treatment or prevention of pain (pain management), comprising administering a therapeutically or prophylactically effective amount of a composition of any one of claims 43-44 to a subject in need thereof.

53. Method for the treatment or prevention of epidermolysis bullosa, comprising administering a therapeutically or prophylactically effective amount of a composition of any one of claims 45-46 to a subject in need thereof.

54. The method of any one of claims 51-53, wherein the subject is a human.

55. Isolated polypeptide comprising an amino acid sequence that shares at least 70, preferably at least 80, more preferably at least 90%, most preferably at least 95% sequence identity with the amino acid sequence set forth in SEQ ID NO:2 over its entire length (dCasRx) and comprises an amino acid substitution in the position corresponding to position 940 of SEQ ID NO-2, preferably 940L and, optionally any one or more of 239A, 244A, 858A, and 863A, using the positional numbering of SEQ ID NO:2.
